(19) United States
(12) Patent Application Publication
(10) Pub. No.: US 2025/0017671 A1
(43) Pub. Date: Jan. 16, 2025

(54) Surgical Robot Positioning System and Related Devices and Methods

(71) Applicant: Virtual Incision Corporation, Lincoln, NE (US)

(72) Inventors: Shane Farritor, Lincoln, NE (US); Nathan Wood, Lincoln, NE (US); Rachael Wagner, Lincoln, NE (US); Parker Durham, Lincoln, NE (US); Lou Cubrich, Lincoln, NE (US); Jay Carlson, Lincoln, NE (US); Mark Reichenbach, Lincoln, NE (US); Robert M. Cicerchia, Westwood, MA (US); Carsten Horn, Fall River, MA (US)

(21) Appl. No.: 18/904,452

(22) Filed: Oct. 2, 2024

Related U.S. Application Data
(63) Continuation of application No. 17/368,255, filed on Jul. 6, 2021, now Pat. No. 12,150,722.
(60) Provisional application No. 63/048,620, filed on Jul. 6, 2020.

(51) Int. Cl. A61B 34/30; A61G 13/10; B25J 9/02; B25J 9/10

(52) U.S. Cl. CPC A61B 34/30; A61G 13/101; B25J 9/02; B25J 9/1035; A61B 2034/302; A61B 2034/303; A61B 2034/305

(57) ABSTRACT

The various embodiments disclosed herein relate to surgical robot positioning systems and devices that aid in the gross positioning of surgical devices during surgical procedures. For example, a gross positioning system for use with a robotic surgical device may include a positioning body, a yaw mechanism operably coupled to the positioning body at a yaw rotational joint, a pitch mechanism operably coupled to the positioning body at a pitch rotational joint, and a plunge mechanism operably coupled to the pitch mechanism, where the plunge mechanism is configured to slide and to be coupleable to the robotic surgical device.

### CROSS-REFERENCE TO RELATED APPLICATION(S)

This application is a Continuation of and claims the benefit of U.S. patent application Ser. No. 17/368,255, filed Jul. 6, 2021 and entitled “Surgical Robot Positioning System and Related Devices and Methods,” which application claims the benefit under 35 U.S.C. § 119(e) to U.S. Provisional Application 63/048,620, filed Jul. 6, 2020 and entitled “Surgical Robot Positioning System and Related Devices and Methods,” the disclosures of each of which are hereby incorporated herein by reference in their entireties.

### FIELD

The various embodiments herein relate to robotic surgical systems, and more specifically to surgical robot positioning systems and devices that aid in the gross positioning of surgical devices during surgical procedures. The combination of a gross positioning system with an in vivo surgical device results in an increase in the degrees of freedom of the in vivo device without increasing the size of the device.

### BACKGROUND

The known positioning systems currently used for robotic surgery are large and cumbersome. For example, the Da Vinci SP Surgical System™ takes up a significant portion of the operating room and creates a crowded space over the surgical site, and the system created by Waseda University has bulky motor housings that create a larger than necessary profile. In a further example, the Raven™ mimics current laparoscopic techniques by inserting a single tool (in contrast to the in vivo robot systems used in the other two systems discussed above).

Certain of these known systems include a known, generic spherical mechanism that can be used to reach the extents of the abdominal cavity of a patient. A “spherical mechanism” is a physical mechanism or software application that can cause all end effector motions to pass through a single point, thereby allowing a surgical system to use long rigid tools that perform procedures through incisions that serve as single pivot points. As an example, both COBRASurge and the Raven have mechanical spherical mechanisms, while Da Vinci has a software-based spherical mechanism.

There is a need in the art for an improved positioning system.

### BRIEF SUMMARY

Discussed herein are various gross positioning systems for use with robotic surgical devices such as in vivo surgical devices.

In Example 1, a gross positioning system for use with a robotic surgical device comprises a positioning body, a yaw mechanism operably coupled to the positioning body at a first rotational joint, a pitch mechanism operably coupled to the positioning body at a second rotational joint, and a plunge mechanism slidably coupled to the pitch mechanism such that the plunge mechanism can move along a length of a plunge axis, wherein the plunge mechanism is configured to be coupleable to the robotic surgical device.

Example 2 relates to the gross positioning system according to Example 1, wherein the yaw mechanism further comprises a motor operably engaged with an output shaft configured to rotate the positioning body around the first rotational joint.

Example 3 relates to the gross positioning system according to Example 2, wherein the yaw mechanism further comprises: a drive gear coupled to the motor, a driven gear operably engaged with the drive gear, a screw coupled to the driven gear, and a wheel is coupled to the output shaft, wherein the wheel is operably engaged with the screw.

Example 4 relates to the gross positioning system according to Example 1, wherein the pitch mechanism further comprises a motor operably engaged with a curved output rail configured to rotate the plunge mechanism around the second rotational joint.

Example 5 relates to the gross positioning system according to Example 4, wherein the pitch mechanism further comprises: a screw coupled to the motor, a wheel operably engaged with the screw, and a rotatable gear operably coupled to the wheel, wherein the rotatable gear is operably engaged with the curved output rail.

Example 6 relates to the gross positioning system according to Example 1, wherein the plunge mechanism further comprises a motor operably engaged with an elongate output rail configured to translationally move the plunge mechanism along the plunge axis.

Example 7 relates to the gross positioning system according to Example 1, wherein the plunge mechanism further comprises a clamp configured to be coupleable to the robotic surgical device.

Example 8 relates to the gross positioning system according to Example 1, wherein a first axis of rotation of the first rotational joint, a second axis of rotation of the second rotational joint, and the plunge axis intersect at a single point of intersection.

Example 9 relates to the gross positioning system according to Example 8, further comprising two or more lasers configured to emit light beams intersecting at the single point of intersection.

Example 10 relates to the gross positioning system according to Example 1, further comprising a controller operably coupled to the gross positioning system and the robotic surgical device, wherein the gross positioning system and robotic surgical device are configured to operate together to position the robotic surgical device within a body cavity of a patient.

In Example 11, a gross positioning system for use with a robotic surgical device comprises a positioning body, a yaw mechanism operably coupled to the positioning body at a first rotational joint, a pitch mechanism operably coupled to the positioning body at a second rotational joint, a plunge mechanism slidably coupled to the pitch mechanism such that the plunge mechanism can move along a length of a plunge axis, wherein the plunge mechanism is configured to translationally move the robotic surgical device along the length of the plunge axis, and the robotic surgical device operably coupled to the plunge mechanism, the robotic surgical device comprising a device body and an arm operably coupled to the device body, the arm comprising an end effector, wherein the robotic surgical device is positionable through an insertion point in a patient such that the arm and at least a portion of the device body is positionable within a body cavity of the patient.

Example 12 relates to the gross positioning system according to Example 11, wherein a first axis of rotation of the first rotational joint, a second axis of rotation of the second rotational joint, and the plunge axis intersect at a single point of intersection.

Example 13 relates to the gross positioning system according to Example 12, wherein the single point of intersection is disposed at some point along a portion of the robotic surgical device.

Example 14 relates to the gross positioning system according to Example 12, wherein the single point of intersection is disposed at an insertion point of a patient and the arm is partially disposed through the single point of intersection.

Example 15 relates to the gross positioning system according to Example 14, wherein the insertion point comprises an incision or a natural orifice.

In Example 16, an external gross positioning system for use with an internal robotic surgical device comprises a support arm, a positioning body operably coupled to the support arm, a yaw mechanism operably coupled to the positioning body at a first rotational joint, a pitch mechanism operably coupled to the positioning body at a second rotational joint, a plunge mechanism slidably coupled to the pitch mechanism such that the plunge mechanism can move along a length of a plunge axis, wherein the plunge mechanism is configured to be coupleable to the internal robotic surgical device, and a single point of intersection of an axis of rotation of the first rotational joint, an axis of rotation of the second rotational joint, and the plunge axis.

Example 17 relates to the external gross positioning system according to Example 16, wherein the support arm further comprises a clamp configured to couple with a bed rail, a rod coupled to the clamp, a first elongate arm operably coupled to the rod at a third rotational joint, and a second elongate arm operably coupled to the first elongate arm at a fourth rotational joint and operably coupled to the positioning body at a fifth rotational joint.

Example 18 relates to the external gross positioning system according to Example 17, wherein the third rotational joint, the fourth rotational joint, and the fifth rotational joint are each configured to rotate around parallel axes.

Example 19 relates to the external gross positioning system according to Example 16, wherein the robotic surgical device comprises at least one arm, wherein the external gross positioning system and robotic surgical device are configured to operate together to position the robotic surgical device within a body cavity of a patient.

Example 20 relates to the external gross positioning system according to Example 19, further comprising a central processing unit operably coupled to the external gross positioning system and the robotic surgical device, wherein the central processing unit comprises software configured to transmit control instructions to the external gross positioning system and the robotic surgical device, and a controller operably coupled to the central processing unit.

In Example 21, a method for performing surgery comprises rotating, at a first rotational joint, a yaw mechanism operably coupled to a positioning body at the first rotational joint, rotating, at a second rotational joint, a pitch mechanism operably coupled to the positioning body at the second rotational joint, and sliding, along a length of a plunge axis, a plunge mechanism operably coupled to the pitch mechanism, wherein a robotic surgical device is configured to slide with the robotic surgical device along the length of the plunge axis.

In Example 22, a method for performing surgery comprises adjusting a first arm of a support arm operably coupled to a positioning body, rotating, at a first rotational joint, a yaw mechanism operably coupled to the positioning body at the first rotational joint, rotating, at a second rotational joint, a pitch mechanism operably coupled to the positioning body at the second rotational joint, sliding, along a length of a plunge axis, a plunge mechanism operably coupled to the pitch mechanism, wherein a robotic surgical device is configured to slide with the robotic surgical device along the length of the plunge axis, and aligning the robotic surgical device at a single point of intersection of an axis of rotation of the first rotational joint, an axis of rotation of the second rotational joint, and the plunge axis.

While multiple embodiments are disclosed, still other embodiments will become apparent to those skilled in the art from the following detailed description, which shows and describes illustrative embodiments. As will be realized, the various implementations are capable of modifications in various obvious aspects, all without departing from the spirit and scope thereof. Accordingly, the drawings and detailed description are to be regarded as illustrative in nature and not restrictive.

### BRIEF DESCRIPTION OF THE DRAWINGS

FIG. 1 is a robotic surgical system, according to one embodiment.

FIG. 2A is a perspective view of a robotic surgical device positioning system coupled to an in vivo robotic device, according to one embodiment.

FIG. 2B is a detailed view of a gross positioning system coupled to an in vivo robotic device that is disposed within a cavity of a patient, according to one embodiment.

FIG. 3A is a detailed perspective view of a gross positioning system, according to one embodiment.

FIG. 3B is another detailed perspective view of a gross positioning system, according to one embodiment.

FIG. 4A is a detailed perspective view of the workspace of a gross positioning system coupled to an in vivo robotic device, according to one embodiment.

FIG. 4B is a detailed side view of a cross section of the workspace of a gross positioning system coupled to an in vivo robotic device of FIG. 4A.

FIG. 5 is a detailed side view of a gross positioning system coupled to an in vivo robotic device with a camera, according to one embodiment.

FIG. 6A is a perspective view of a yaw mechanism and the support arm of a gross positioning system, according to one embodiment.

FIG. 6B is an exploded cross-sectional view of the yaw mechanism of a gross positioning system of FIG. 6A.

FIG. 7A is a perspective view of a yaw mechanism of a gross positioning system, according to one embodiment.

FIG. 7B is a detailed view of the components of the yaw mechanism of FIG. 7A.

FIG. 7C is a side cross-sectional view of the yaw mechanism of FIG. 7A.

FIG. 7D is a front cross-sectional view of the yaw mechanism of FIG. 7A.

FIG. 8A is a perspective view of a pitch mechanism of a gross positioning system, according to one embodiment.

FIG. 8B is a detailed view of the components of the pitch mechanism of FIG. 8A.

FIG. 8C is a detailed view of the rail of the pitch mechanism of FIG. 8A connected to a plunge mechanism.

FIG. 9A is a perspective view of a plunge mechanism of a gross positioning system, according to one embodiment.

FIG. 9B is a detailed view of the components of the plunge mechanism of FIG. 9A.

FIG. 10 is a robotic surgical device positioning system, according to one embodiment.

FIG. 11A is a detailed view of a clamping mechanism of a gross positioning system in an unlocked position, according to one embodiment.

FIG. 11B is a detailed view of the clamping mechanism of FIG. 11A in a locked position.

FIG. 11C is a detailed view of the clamping mechanism of FIG. 11A in a locked position and a support arm of the gross positioning system.

FIG. 11D is a cross-sectional view of the clamping mechanism of FIG. 11A in a locked position.

FIG. 12 is a robotic surgical device positioning system with lasers, according to one embodiment.

FIG. 13A is a perspective view of a pitch mechanism of a gross positioning system, according to one embodiment.

FIG. 13B is a perspective view of a plunge mechanism of a gross positioning system, according to one embodiment.

FIG. 14A is a perspective view of a gross positioning system, according to one embodiment.

FIG. 14B is another perspective view of the gross positioning system of FIG. 14A.

FIG. 15A is a detailed view of a rail of a plunge mechanism of a gross positioning system, according to one embodiment.

FIG. 15B is another detailed view of the rail of the plunge mechanism of FIG. 15A.

FIG. 16 is a perspective view of a robotic surgical device positioning system coupled to an in vivo robotic device, according to one embodiment.

FIG. 17A is a detailed perspective view of a gross positioning system, according to one embodiment.

FIG. 17B is another detailed perspective view of the gross positioning system, according to one embodiment.

FIG. 18A is a detailed perspective view of the workspace of a gross positioning system coupled to an in vivo robotic device, according to one embodiment.

FIG. 18B is a detailed side view of a cross section of the workspace of a gross positioning system coupled to an in vivo robotic device of FIG. 18A.

FIG. 19A is a perspective view of a yaw mechanism and the support arm of a gross positioning system, according to one embodiment.

FIG. 19B is an exploded cross-sectional view of the yaw mechanism of a gross positioning system of FIG. 19A.

FIG. 20A is a perspective view of a yaw mechanism of a gross positioning system, according to one embodiment.

FIG. 20B is a detailed view of the components of the yaw mechanism of FIG. 20A.

FIG. 20C is a side detailed view of the components of the yaw mechanism of FIG. 20A.

FIG. 20D is a side cross-sectional view of the yaw mechanism of FIG. 20A.

FIG. 21A is a perspective view of a pitch mechanism of a gross positioning system, according to one embodiment.

FIG. 21B is a detailed view of the components of the pitch mechanism of FIG. 21A.

FIG. 21C is a detailed view of the rail of the pitch mechanism connected to a plunge mechanism.

FIG. 22A is a perspective view of a plunge mechanism of a gross positioning system, according to one embodiment.

FIG. 22B is a detailed view of the components of the plunge mechanism of FIG. 22A.

FIG. 23 is a robotic surgical device positioning system, according to one embodiment.

FIG. 24A is a perspective view of a clamping mechanism of a gross positioning system, according to one embodiment.

FIG. 24B is a detailed view of the clamping mechanism of FIG. 24A.

FIG. 25 is a robotic surgical device positioning system with lasers, according to one embodiment.

FIG. 26A is a perspective view of a pitch mechanism of a gross positioning system, according to one embodiment.

FIG. 26B is a perspective view of a plunge mechanism of a gross positioning system, according to one embodiment.

### DETAILED DESCRIPTION

The various embodiments disclosed or contemplated herein relate to a surgical robot positioning system that includes a passive support arm and a gross positioning robotic device. A dexterous in vivo surgical robotic device is coupleable to the gross positioning robotic device such that the positioning system can be used for global orientation of the surgical robotic device within the cavity of a patient as described in further detail herein.

The various gross positioning system implementations disclosed or contemplated herein can be used to automatically grossly position a surgical device inside a cavity of a patient. “Gross positioning,” as used herein, is intended to mean general positioning of an entire moveable surgical device (in contrast to precise movement and placement of the specific components of such a device, such as an arm or end effector). In known robotic surgical systems, the gross positioning of those devices during a surgical procedure can be a challenging task. Further, minimally invasive surgical procedures (using either robotic or non-robotic systems) frequently require a surgical technician to reposition the surgical equipment, such as a laparoscope. Such gross repositioning takes time and additional effort. In some cases, the surgical technician is a junior medical student who is not fully trained in laparoscopy. As a result, the repositioning instructions from the surgeon often result in an obstructed and/or fogged view of the surgical site, requiring additional cognitive resources from the surgeon. For example, the Da Vinci@ system as well as known single incision surgical devices often require timely manual repositioning of the patient, the robotic system, or both while performing complicated procedures.

The various gross positioning systems contemplated herein aid in the gross repositioning of surgical devices throughout the procedure without additional intervention or manual repositioning from the surgical staff. The surgical devices may include, for example, any surgical devices that have a device body, rod, or tube configured to be positioned through an incision and at least one robotic arm coupled to or positioned through the device body or tube that is positioned entirely within the cavity of the patient. The gross positioning system embodiments can control the degrees of freedom, azimuth and elevation angle, and roll and translation about the axis of insertion of laparoscopic surgical tools, including robotic laparoscopic surgical tools. As a result, the gross positioning system embodiments disclosed and contemplated herein can grossly position a surgical device through an incision, port, or orifice (including a natural orifice) into a patient cavity, such as the abdominal cavity, with high manipulability, reducing the operative time and stress induced upon the surgical staff. The combination of the external gross positioning system with the internal surgical device system will allow the degrees of freedom of the internal system to effectively increase without increasing the size of the surgical robot/device.

In one implementation, the various systems and devices described and contemplated herein can be used with any single site surgical device or system with an available external positioning fixture, such as a protruding body, rod, tube, or magnetic handle. Further, it is understood that the various embodiments of positioning systems disclosed herein can be used with any other known medical devices, systems, and methods that are positioned through an incision, port, or orifice (including a natural orifice). For example, the various embodiments disclosed herein may be used with any of the medical devices and systems disclosed in U.S. Pat. No. 8,968,332 (issued on Mar. 3, 2015 and entitled “Magnetically Coupleable Robotic Devices and Related Methods”), U.S. Pat. No. 8,834,488 (issued on Sep. 16, 2014 and entitled “Magnetically Coupleable Surgical Robotic Devices and Related Methods”), U.S. Pat. No. 10,307,199 (issued on Jun. 4, 2019 and entitled “Robotic Surgical Devices and Related Methods”), U.S. Pat. No. 9,579,088 (issued on Feb. 28, 2017 and entitled “Methods, Systems, and Devices for Surgical Visualization and Device Manipulation”), U.S. Patent Application 61/030,588 (filed on Feb. 22, 2008), U.S. Pat. No. 8,343,171 (issued on Jan. 1, 2013 and entitled “Methods and Systems of Actuation in Robotic Devices”), U.S. Pat. No. 8,828,024 (issued on Sep. 9, 2014 and entitled “Methods and Systems of Actuation in Robotic Devices”), U.S. Pat. No. 9,956,043 (issued on May 1, 2018 and entitled “Methods and Systems of Actuation in Robotic Devices”), U.S. patent application Ser. No. 15/966,606 (filed on Apr. 30, 2018 and entitled “Methods, Systems, and Devices for Surgical Access and Procedures”), U.S. patent application Ser. No. 12/192,663 (filed on Aug. 15, 2008 and entitled “Medical Inflation, Attachment, and Delivery Devices and Related Methods”), U.S. patent application Ser. No. 15/018,530 (filed on Feb. 8, 2016 and entitled “Medical Inflation, Attachment, and Delivery Devices and Related Methods”), U.S. Pat. No. 8,974,440 (issued on Mar. 10, 2015 and entitled “Modular and Cooperative Medical Devices and Related Systems and Methods”), U.S. Pat. No. 8,679,096 (issued on Mar. 25, 2014 and entitled “Multifunctional Operational Component for Robotic Devices”), U.S. Pat. No. 9,179,981 (issued on Nov. 10, 2015 and entitled “Multifunctional Operational Component for Robotic Devices”), U.S. Pat. No. 9,883,911 (issued on Feb. 6, 2018 and entitled “Multifunctional Operational Component for Robotic Devices”), U.S. patent application Ser. No. 15/888,723 (filed on Feb. 5, 2018 and entitled “Multifunctional Operational Component for Robotic Devices”), U.S. Pat. No. 8,894,633 (issued on Nov. 25, 2014 and entitled “Modular and Cooperative Medical Devices and Related Systems and Methods”), U.S. Pat. No. 8,968,267 (issued on Mar. 3, 2015 and entitled “Methods and Systems for Handling or Delivering Materials for Natural Orifice Surgery”), U.S. Pat. No. 9,060,781 (issued on Jun. 23, 2015 and entitled “Methods, Systems, and Devices Relating to Surgical End Effectors”), U.S. Pat. No. 9,757,187 (issued on Sep. 12, 2017 and entitled “Methods, Systems, and Devices Relating to Surgical End Effectors”), U.S. Pat. No. 10,350,000 (issued on Jul. 16, 2019 and entitled “Methods, systems, and devices relating to surgical end effectors”), U.S. patent application Ser. No. 16/512,510 (filed on Jul. 16, 2019 and entitled “Methods, Systems, and Devices Relating to Surgical End Effectors”), U.S. Pat. No. 9,089,353 (issued on Jul. 28, 2015 and entitled “Robotic Surgical Devices, Systems, and Related Methods”), U.S. Pat. No. 10,111,711 (issued on Oct. 30, 2018 and entitled “Robotic Surgical Devices, Systems, and Related Methods”), U.S. patent application Ser. No. 16/123,619 (filed on Sep. 6, 2018 and entitled “Robotic Surgical Devices, Systems and Related Methods”), U.S. Pat. No. 9,770,305 (issued on Sep. 26, 2017 and entitled “Robotic Surgical Devices, Systems, and Related Methods”), U.S. patent application Ser. No. 15/661,147 (filed on Jul. 27, 2017 and entitled “Robotic Devices with On Board Control & Related Systems & Devices”), U.S. patent application Ser. No. 13/833,605 (filed on Mar. 15, 2013 and entitled “Robotic Surgical Devices, Systems, and Related Methods”), U.S. patent application Ser. No. 13/738,706 (filed on Jan. 10, 2013 and entitled “Methods, Systems, and Devices for Surgical Access and Insertion”), U.S. patent application Ser. No. 14/661,465 (filed on Mar. 18, 2015 and entitled “Methods, Systems, and Devices for Surgical Access and Insertion”), U.S. patent application Ser. No. 15/890,860 (filed on Feb. 7, 2018 and entitled “Methods, Systems, and Devices for Surgical Access and Insertion”), U.S. Pat. No. 9,498,292 (issued on Nov. 22, 2016 and entitled “Single Site Robotic Devices and Related Systems and Methods”), U.S. Pat. No. 10,219,870 (issued on Mar. 5, 2019 and entitled “Single site robotic device and related systems and methods”), U.S. patent application Ser. No. 16/293,135 (filed Mar. 3, 2019 and entitled “Single Site Robotic Device and Related Systems and Methods”), U.S. Pat. No. 9,010,214 (issued on Apr. 21, 2015 and entitled “Local Control Robotic Surgical Devices and Related Methods”), U.S. Pat. No. 10,470,828 (issued on Nov. 12, 2019 and entitled “Local Control Robotic Surgical Devices and Related Methods”), U.S. patent application Ser. No. 16/596,034 (filed on Oct. 8, 2019 and entitled “Local Control Robotic Surgical Devices and Related Methods”), U.S. Pat. No. 9,743,987 (issued on Aug. 29, 2017 and entitled “Methods, Systems, and Devices Relating to Robotic Surgical Devices, End Effectors, and Controllers”), U.S. patent application Ser. No. 15/687,787 (filed on Aug. 28, 2017 and entitled “Methods, Systems, and Devices Relating to Robotic Surgical Devices, End Effectors, and Controllers”), U.S. Pat. No. 9,888,966 (issued on Feb. 13, 2018 and entitled “Methods, Systems, and Devices Relating to Force Control Surgical Systems”), U.S. patent application Ser. No. 15/894,489 (filed on Feb. 12, 2018 and entitled “Methods, Systems, and Devices Relating to Force Control Surgical Systems”), U.S. patent application Ser. No. 14/212,686 (filed on Mar. 14, 2014 and entitled “Robotic Surgical Devices, Systems, and Related Methods”), U.S. patent application Ser. No. 14/334,383 (filed on Jul. 17, 2014 and entitled “Robotic Surgical Devices, Systems, and Related Methods”), U.S. patent application Ser. No. 14/853,477 (filed on Sep. 14, 2015 and entitled “Quick-Release End Effectors and Related Systems and Methods”), U.S. patent application Ser. No. 16/504,793 (filed on Jul. 8, 2019 and entitled “Quick-Release End Effectors and Related Systems and Methods”), U.S. Pat. No. 10,376,322 (issued on Aug. 13, 2019 and entitled “Robotic Device with Compact Joint Design and Related Systems and Methods”), U.S. patent application Ser. No. 16/538,902 (filed on Aug. 13, 2019 and entitled “Robotic Device with Compact Joint Design and Related Systems and Methods”), U.S. patent application Ser. No. 15/227,813 (filed on Aug. 3, 2016 and entitled Robotic Surgical Devices, System and Related Methods”) U.S. patent application Ser. No. 15/599,231 (filed on May 18, 2017 and entitled “Robotic Surgical Devices, Systems, and Related Methods”), U.S. patent application Ser. No. 15/687,113 (filed on Aug. 25, 2017 and entitled “Quick-Release End Effector Tool Interface”), U.S. patent application Ser. No. 15/691,087 (filed on Aug. 30, 2017 and entitled “Robotic Device with Compact Joint Design and an Additional Degree of Freedom and Related Systems and Methods”), U.S. patent application Ser. No. 15/826,166 (filed on Nov. 29, 2017 and entitled “User controller with user presence detection and related systems and methods”), U.S. patent application Ser. No. 15/842,230 (filed on Dec. 14, 2017 and entitled “Releasable Attachment Device for Coupling to Medical Devices and Related Systems and Methods”), U.S. patent application Ser. No. 16/144,807 (filed on Sep. 27, 2018 and entitled “Robotic Surgical Devices with Tracking Camera Technology and Related Systems and Methods”), U.S. patent application Ser. No. 16/241,263 (filed on Jan. 7, 2019 and entitled “Single-Manipulator Robotic Device With Compact Joint Design and Related Systems and Methods”), U.S. Pat. No. 7,492,116 (filed on Oct. 31, 2007 and entitled “Robot for Surgical Applications”), U.S. Pat. No. 7,772,796 (filed on Apr. 3, 2007 and entitled “Robot for Surgical Applications”), and U.S. Pat. No. 8,179,073 (issued on May 15, 2011, and entitled “Robotic Devices with Agent Delivery Components and Related Methods”), all of which are hereby incorporated herein by reference in their entireties.

Certain device and system implementations disclosed in the applications listed above can be positioned within a body cavity of a patient, or a portion of the device can be placed within the body cavity, in combination with a positioning system such as any of the embodiments disclosed or contemplated herein. An “in vivo device” as used herein means any device that can be positioned, operated, or controlled at least in part by a user while being positioned within a body cavity of a patient, including any device that is coupled to a support component such as a rod, tube, body, or other such component that is disposed through an opening or orifice of the body cavity, also including any device positioned substantially against or adjacent to a wall of a body cavity of a patient, further including any such device that is internally actuated (having no external source of motive force), and additionally including any device that may be used laparoscopically or endoscopically during a surgical procedure. As used herein, the terms “robot,” and “robotic device” shall refer to any device that can perform a task either automatically or in response to a command.

In certain implementations, any robotic device that is coupleable to the various positioning system embodiments disclosed or contemplated herein can be positioned through an insertion port. The insertion port can be a known, commercially-available flexible membrane (referred to herein as a “gelport”) placed transabdominally to seal and protect the abdominal incision. This off-the-shelf component is the same device or substantially the same device that is used in substantially the same way for Hand-Assisted Laparoscopic Surgery (HALS). The only difference is that the arms of the robotic device according to the various embodiments herein are inserted into the abdominal cavity through the insertion port rather than the surgeon's hand. The robotic device body, rod, or tube seals against the insertion port when it is positioned therethrough, thereby maintaining insufflation pressure. The port is single-use and disposable. Alternatively, any known port can be used. In further alternatives, the various devices that can be used in combination with the various positioning system embodiments herein can be inserted through an incision without a port or through a natural orifice.

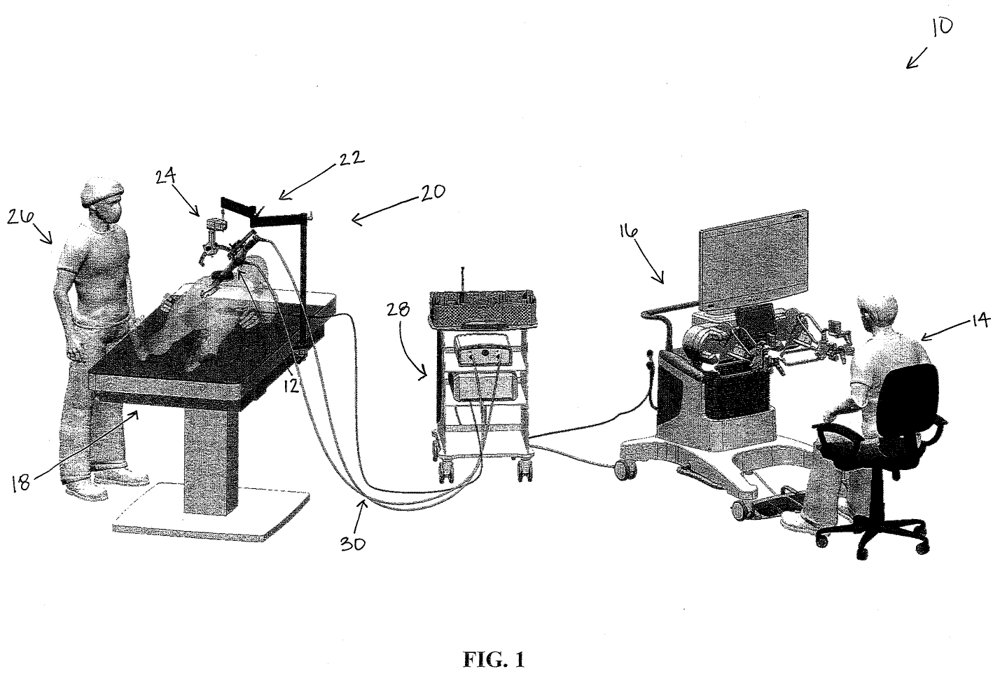

FIG. 1 depicts one embodiment of a robotic surgical system 10 having several components that will be described in additional detail below. The components of the various positioning system implementations disclosed or contemplated herein can be used with a full surgical system 10 that includes an external control console 16 and a robotic surgical device 12. In accordance with the implementation of FIG. 1, the robotic surgical device 12 is shown mounted to the operating table 18 (or a rail thereof) via a robot positioning system 20 according to one embodiment as described in additional detail below. The robot positioning system 20 has a passive support arm 22 and a robotic positioning device 24 coupled to the arm 22. The support arm 22 is coupled to the operating table 18 and the robotic device 12 is coupleable to the robotic positioning device 24. The system 10 can be, in certain implementations, operated by the surgeon 14 at the console 16 and one surgical assistant 26 positioned at the operating table 18. That is, the surgeon 14 at the console 16 can control both the robotic device 12 and the gross positioning robotic device 24, and the surgical assistant 26 can control the remaining system 10 components (e.g., passive support arm 22). Alternatively, one surgeon 14 can operate the entire system 10. In a further alternative, three or more people can be involved in the operation of the system 10. It is further understood that the surgeon (or user) 14 can be located at a remote location in relation to the operating table 18 such that the surgeon 14 can be in a different city or country or on a different continent from the patient on the operating table 18. The console 16 can be any console as disclosed in any of the various patents and/or applications incorporated by reference above. Alternatively, the console 16 can be any known console for operating a robotic surgical system or device.

In this specific implementation, the robotic device 12 is connected to the interface pod and electrosurgical unit 28 via connection cables 30. Further, the gross positioning robotic device 24 is also coupled to the interface pod and electrosurgical unit 28 via the connection cables 30. Alternatively, any wired or wireless connection configuration can be used. Further, the interface pod and electrosurgical unit 28 is coupled to the console 16 as shown (and alternatively can be coupled via any known wired or wireless connection). In certain implementations, the system 10 can also interact with other devices during use such as auxiliary monitors, etc.

According to various embodiments, the gross positioning robotic device 24 of the positioning system 20 can dock or otherwise couple with the surgical robotic device 12 and control the position of the workspace of the device 12 by supporting and moving the surgical robotic device 12 during a surgical procedure. This allows the surgeon 14 (and the assistant 26) to have complete control of the robotic device 12 with respect to the target surgical area (the target cavity of the patient).

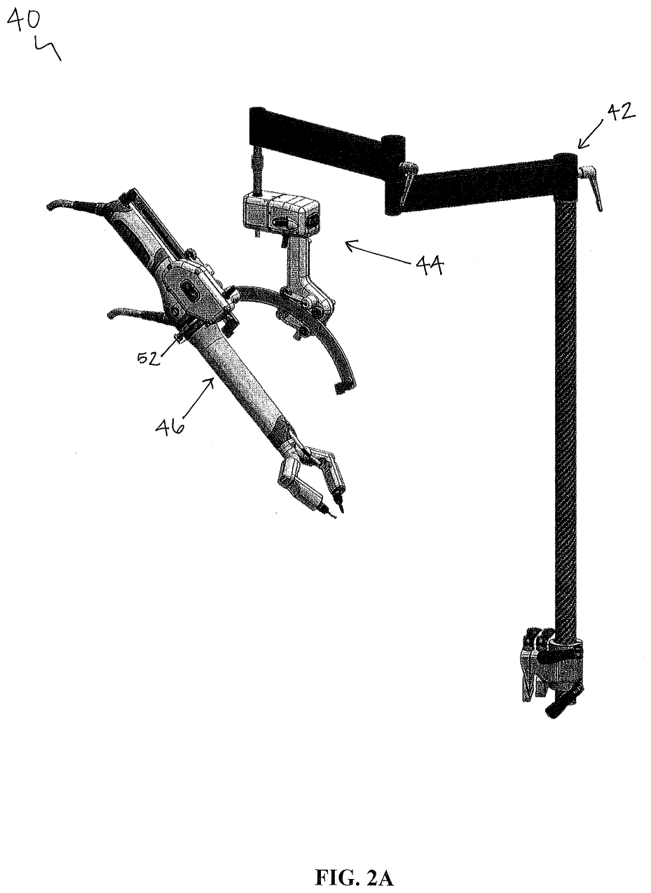

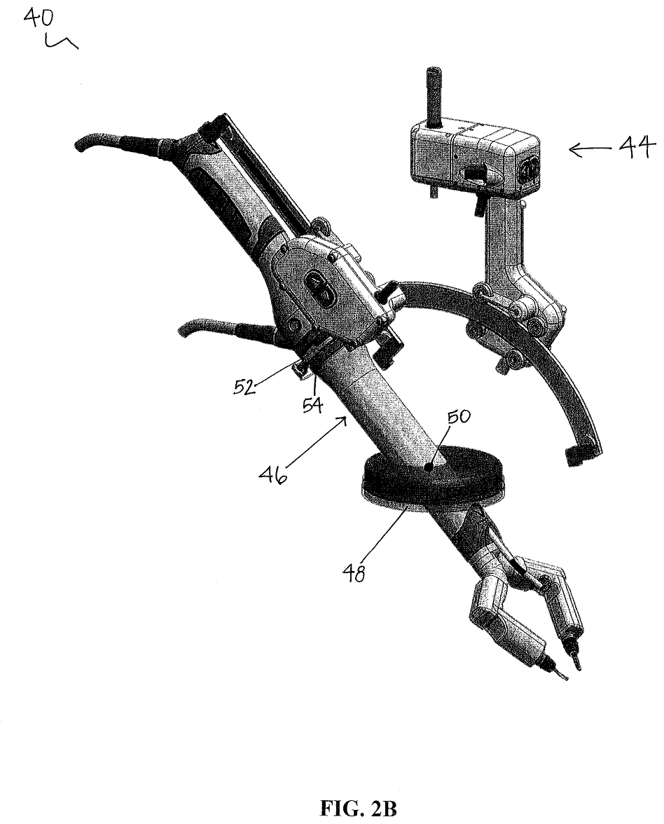

One embodiment of a robotic surgical device positioning system 40 is depicted in FIGS. 2A and 2B. The system 40 includes a passive support arm 42 and a gross robot positioning device 44 rotatably coupled to the arm 42. Further, any known robotic device 46, which in this specific exemplary implementation is represented by the surgical device 46 as shown, can be removably coupled to the positioning device 44 such that the robotic surgical device 46 is disposed through an opening, orifice, incision, or port into the target cavity of the patient. In this specific embodiment, the robotic surgical device 46 is disposed through a port 48 as best shown in FIG. 2B. In certain embodiments, the port 48 is a gelport 48.

The gross positioning device 44 in this implementation—and various other embodiments as disclosed or contemplated herein—is a 3 degree-of-freedom (“DOF”) robotic remote center-of-motion (RCM) mechanism. It is understood that an RCM is the point about which a rotational joint rotates and further that an RCM mechanism is a device where all the kinematic joints move through the same RCM point. For the various gross positioning devices herein (including the robot positioning device 44), the RCM point is within the workspace of the robot positioning device 44 such that, while the end effector of the robotic surgical device 46 can still desirably be manipulated, there is a point of no relative motion with respect to the rest of the mechanism. More specifically, in many implementations the RCM is approximately located at the incision, port, or orifice during surgical use. For example, in the specific embodiment as best shown in FIGS. 2B, the RCM point 50 is positioned at the port 48. As such, there is no relative motion at this patient-device interface that could cause harm to the patient, while still allowing the robotic surgical device 46 full access to the target surgical site (a cavity within the patient).

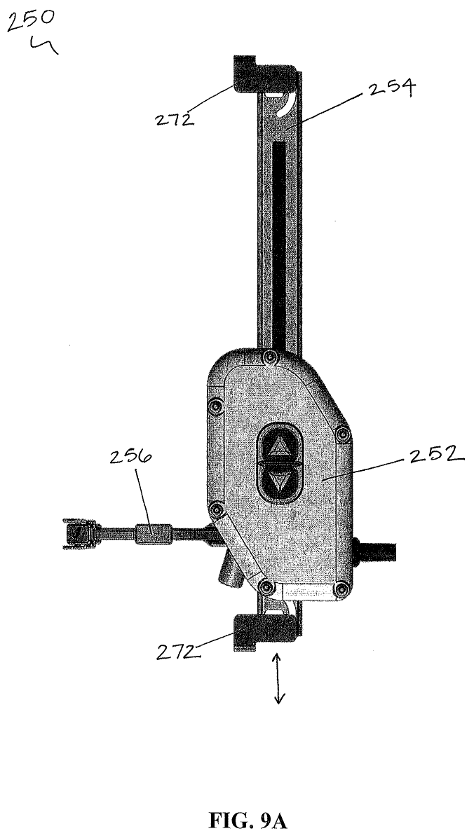

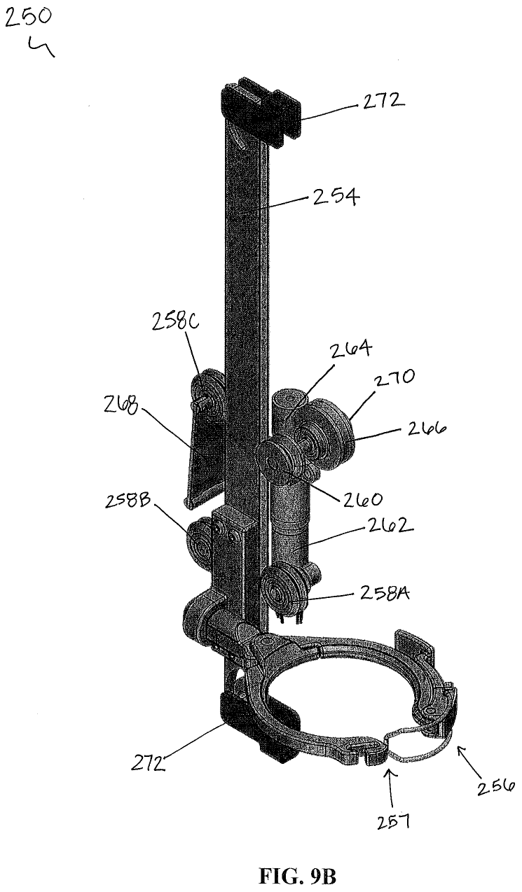

The surgical robotic device 46 can be docked or otherwise removably coupled to the gross positioning device 44 via a connecting clamp 52, as best shown in FIGS. 2A-2B, 3B, and 9B. In this particular embodiment, the clamp 52 is coupleable to the robot positioning device 44 at a specific location on the robotic surgical device 44 having a recessed area 54 (e.g., a clamping groove) around the external surface of the device 44 such that the clamp 52 can easily be disposed within the recessed area 54. For example, as shown in FIG. 9B, a similar clamp 256 embodiment has a latch mechanism 257. Alternatively, any known coupling feature or mechanism can be used. In this embodiment, when the surgical robotic device 46 is docked with the gross positioning device 44, it does not move or rotate with respect to the clamp 52. The attachment mechanism 52 is easily and quickly disengaged when desired by the user.

As discussed above, the port 48 as best shown in FIG. 2B can come in various forms. One embodiment would be a gelport that includes a gel like substance that would seal around the circumference of the robot, to maintain insufflation, while still allowing the robot to move. Another port would use air flow to maintain patient insufflation. Others might use various types of mechanical seals such as diaphragm, duck bill, O-ring or other types of seals or ports. Any known port that can maintain a fluidic seal can be used.

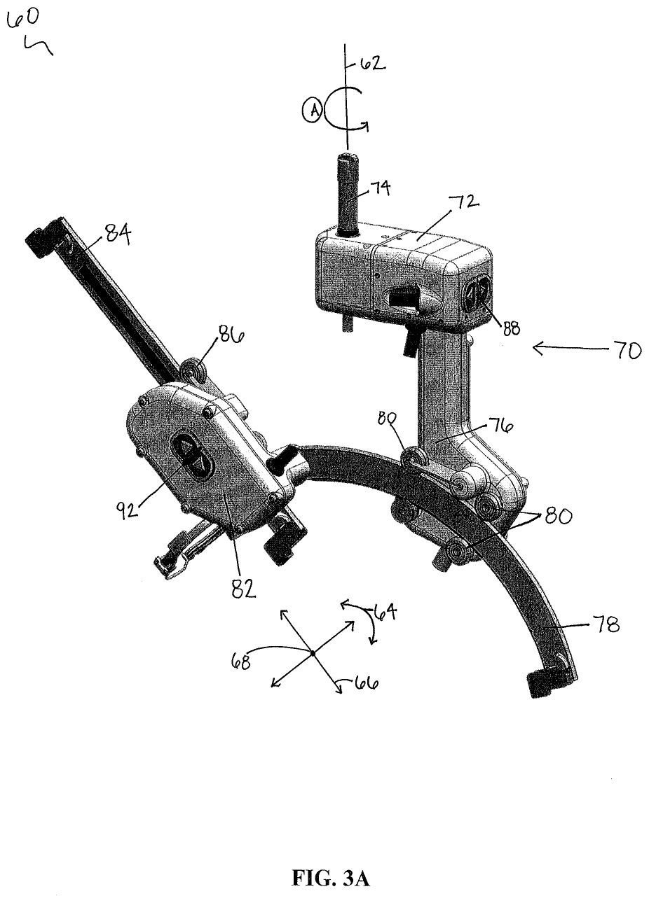

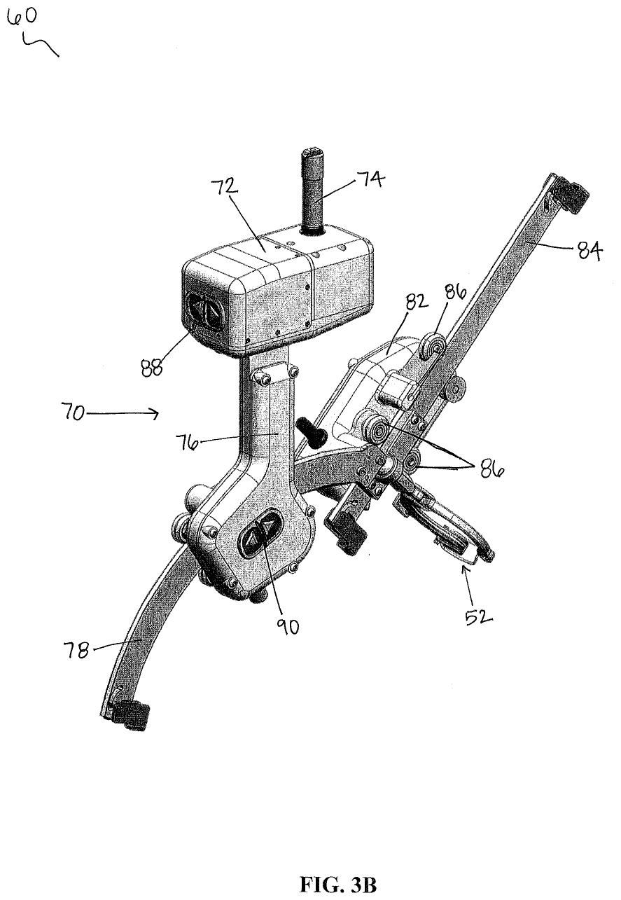

According to another embodiment of a gross positioning device 60 as shown in FIGS. 3A and 3B, the positioning device 60 has three joints: yaw (joint 1) 62, pitch (joint 2) 64, and plunge (joint 3) 66. Yaw 62 and pitch 64 rotate about the RCM, and plunge 66 moves through the RCM 68. In this embodiment, each joint is fully decoupled and is controlled independently. Further, in certain embodiments, motors and motor controllers (not shown) are co-located at each joint 62, 64, 66. In addition, it is understood that each joint 62, 64, 66 can be, but does not have to be, backdriveable.

The yaw joint 62 originates from the body 70 of the gross robot positioning device 60, and more specifically in the yaw mechanism structure 72. More specifically, a rotatable yaw output shaft 74 extends from the yaw mechanism structure 72 and constitutes the yaw joint 62. As such, rotation of the output shaft 74 creates the yaw motion about the yaw joint 62. The yaw actuator 88 may be actuated to adjust the yaw orientation of the robot positioning device 60, for example, to adjust the yaw mechanism structure 72 and body 70 to the left or the right as shown by arrow A. That is, the yaw actuator 88 can be actuated to cause the output shaft 74 to rotate and thereby cause the structure 72 and body 70 to rotate in either direction as desired. Additionally, the rotation of the body 70 also rotates the pitch mechanism 76 and the plunge mechanism 82.

The pitch joint 64 also originates from the body 70 of the device 60, and more specifically in the pitch mechanism structure 76. More specifically, an output rail 78 is operably coupled to the pitch mechanism structure 76 via rotatable bearings 80 (e.g., grooved rotatable bearings) such that movement of the output rail 78 in relation to the pitch mechanism structure 76 (as described in detail below) creates the pitch joint 64. In some examples, the rotatable bearings 80 engage with the corresponding geometry of the edges of the output rail 78. As such, actuation of the output rail 78 to move in one direction or the other along the bearings 80 creates the pitch at the pitch joint 64. As best shown in FIG. 3B, the pitch actuator 90 may be actuated to adjust the pitch orientation of the plunge mechanism structure 82. That is, the pitch actuator 90 can be actuated to cause the output rail 78 to move in the desired direction to cause the structure 82 to move as desired.

The plunge joint 66 originates from the plunge mechanism structure 82, which is operably coupled to the output rail 78. More specifically, an extendable rail 84 is operably coupled to the plunge mechanism structure 82 via rotatable bearings 86 (as best shown according to one example in FIGS. 3B and 9A-9B below). In some examples, the rotatable bearings 86 engage with the corresponding geometry of the edges of the extendable rail 84. As such, extension of the extendable rail 84 in the distal direction creates the plunge at the plunge joint 66. The plunge actuator 92 may be actuated to adjust the position of the extendable rail 84 along the plunge joint. That is, the plunge actuator 92 can be actuated to cause the extendable rail 84 to move in the desired direction to cause any device attached thereto to move as desired. Thus, movement of the extendable rail 84 will also move the clamp 52 and a device 46, if docked.

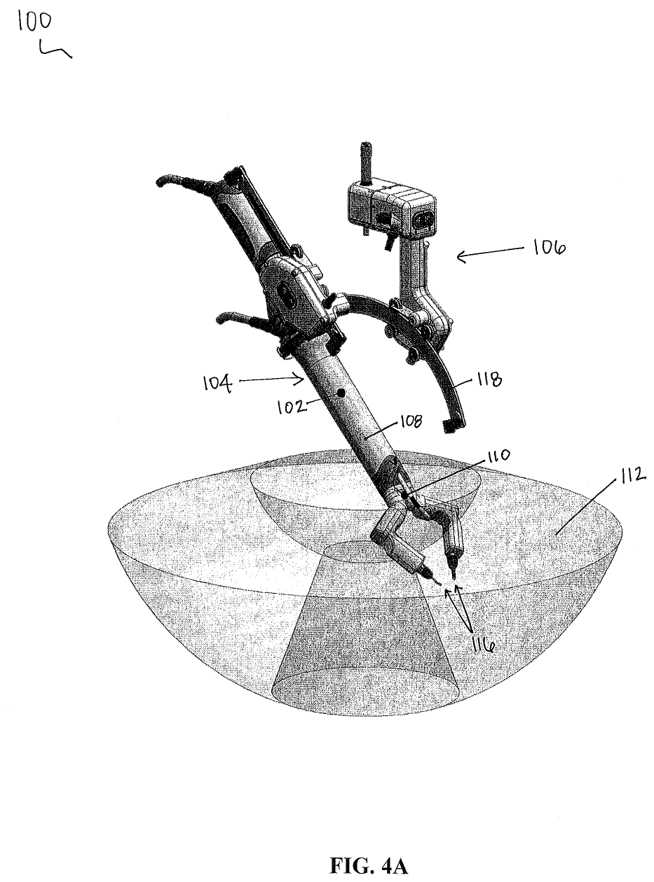

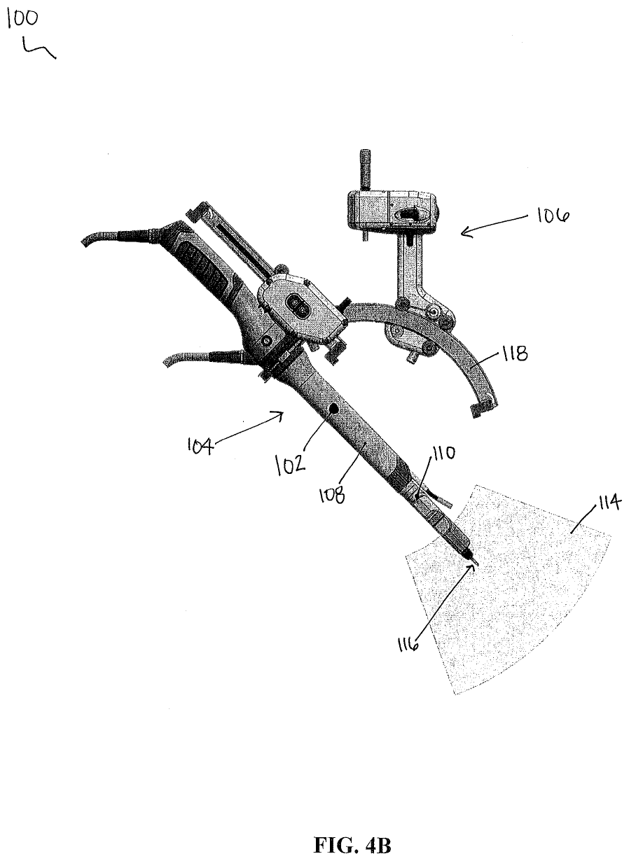

As best shown in FIGS. 4A and 4B, another implementation of a device positioning system 100 has an RCM 102 that is located at the center of the circular pitch arc as at least partially defined by the curved rail 118. Alternatively, the RCM 102 can be disposed in any known position in relation to the gross positioning device 106. When a robotic surgical device—such as device 104—is docked (or otherwise coupled) to the gross robot positioning device 106, the RCM 102 is disposed within the elongate body (or tube) 108 of the robotic surgical device 104 and approximately colinear with its kinematic origin 110, as shown. FIG. 4A also depicts the workspace 112 of the robot positioning device 106 in the context of the robotic surgical device 104. The gross positioning device 106 can be used to move the kinematic origin 110 and thus, the robotic surgical device 104 such that the end effectors 116 can be moved to any location within the workspace 112 (e.g., a toroidal workspace). The cross-section of the workspace 112, which is governed by pitch and plunge, is an annulus sector 114 as best shown in FIG. 4B.

Returning to FIG. 4A, the yaw joint (such as joint 62 at the output shaft 74 as discussed in detail above with respect to FIG. 3A) is a rotational joint and can be articulated over a sweep angle of at least 165 degrees. In the embodiment of FIG. 4A, the workspace 112 depicts rotation around the output shaft 74 (yaw) with 360 degrees of travel, meaning the gross positioning device 106 can, according to certain embodiments, rotate endlessly in either direction (as cabling adjustment permits). The pitch joint (such as joint 64 created by the rail 78 as discussed in detail above with respect to FIG. 3A) allows rotation about the RCM 102 with motion along the output pitch rail 118. For pitch, according to certain implementations, a 50-degree arc can be traversed, with pitch angles approximately between 20 and 70 degrees from vertical. In some alternative embodiments, a 40-degree arc can be traversed, with pitch angles approximately between 20 and 60 degrees from vertical. In addition, the plunge joint (such as joint 66 created by rail 84 as discussed in detail above with respect to FIG. 3A) is a translational joint and can be translated a total length of at least 100 cm, according to various embodiments.

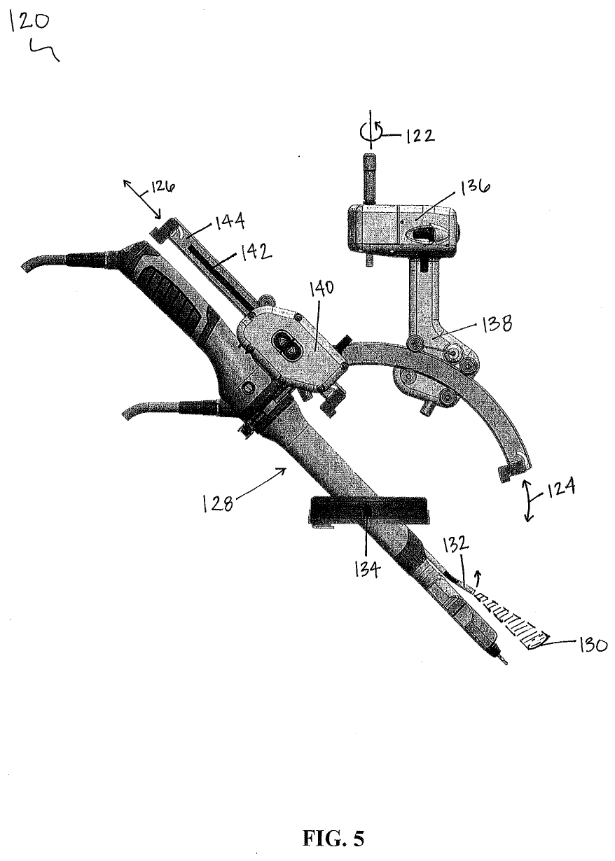

In the embodiment of the robotic gross positioning device 120 depicted in FIG. 5, each joint 122 (yaw), 124 (pitch), and 126 (plunge) can be moved independently, or they can be moved together in any combination and at any rate. Often, the robotic surgical device 128 is repositioned with respect to the frame of view 130 of the camera 132 on the device 128. Because the camera 132 is robotically articulated in this implementation, its field of view 130 may not always be coaxial with the RCM 134. Therefore, sometimes a combination of the joints 122, 124, and 126 must be articulated to move the robotic surgical device 128 in the camera frame 130 as desired, like in the example shown.

The pitch 124 and plunge 126 joints can, in certain embodiments, have precise absolute position control, and can articulate the full workspace allowed by the gross robot positioning device 120 configuration in most surgical situations. It is understood that absolute position sensing can be achieved with encoders (not shown) on the motors (not shown) used to drive the pitch 124 and plunge 126 stages. Alternatively, a vision-based system that reads markings on the drive rails can be used for absolute position sensing. In a further alternative, absolute position sensing in pitch can be accomplished using a pair of inertial measurement units (IMU), with one IMU mounted normal to the yaw joint 122 within the yaw mechanism structure 136 and a second IMU mounted normal to the translational axis of the plunge joint 126 within the plunge mechanism structure 140. Each IMU may be configured to measure the direction of the gravitational acceleration vector relative to the sensor's normal vector. The absolute pitch angle may then be determined (e.g., in real time or on a delay) by calculating the difference between the two IMU readings. For absolute position sensing along the translational axis of the plunge joint 126, a strip 142 (e.g., a Linear Magnetic Scale Nonius Strip) may be located on or embedded in the plunge rail 144. In some cases, the position of this strip may be determined through the use of a pair of anisotropic magnetoresistance sensors mounted on the inside of the plunge mechanism structure 140 proximate to the strip 142. Alternatively, any known sensors or mechanisms can be used to achieve absolute position control.

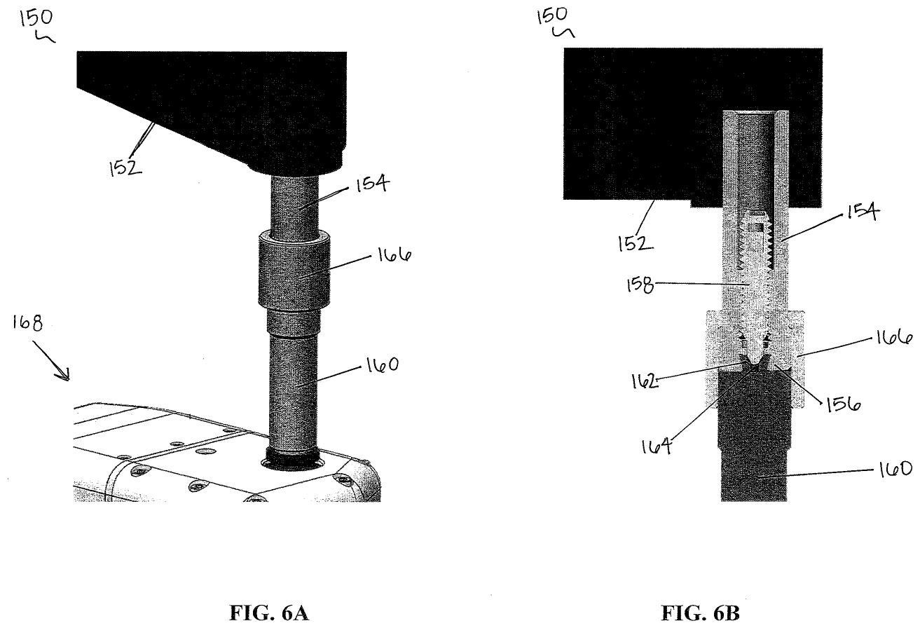

FIGS. 6A and 6B depict one exemplary embodiment of yaw mechanism 150, including an output shaft 160 (similar to the output shaft 74 discussed above) coupled to a connection shaft 154 of a support arm 152. FIG. 6A depicts a perspective view of the output shaft 160 coupled to the connection shaft 154, while FIG. 6B shows a cross-sectional view of the output shaft 160 coupled to the connection shaft 154. In this specific implementation, the coupling and uncoupling of the output shaft 160 and the connection shaft 154 is a relatively fast and simple way to attach/detach the gross positioning device 168 to/from the support arm 152 at the output shaft 160, according to one embodiment.

As shown in FIG. 6B, the output shaft 160 has a male dovetail feature 162 that slides into a female dovetail feature 156 of the connection shaft 154 until a spring-loaded ball detent 158 engages with a center drilled hole 164 of the output shaft 160. This ball detent 158 may preload the connection of the male and female dovetail features 162, 156 and align the output shaft 160 coaxially with the connection shaft 154. To complete the connection, a sleeve 166 is slid down over the connection of the output shaft 160 and the connection shaft 154 to secure the connection and prevent unintended decoupling. The connection supports the weight of the device(s) and prevents rotational motion between the output shaft 160 and the connection shaft 154, while the sleeve 166 prevents relative translation. Alternatively, any other known quick connection mechanisms (such as, for example, the mechanism shown in FIGS. 19A and 19B) can be used for easy docking and undocking of the gross positioning device 168 to the robot support arm 152, such as for cleaning and sterilization.

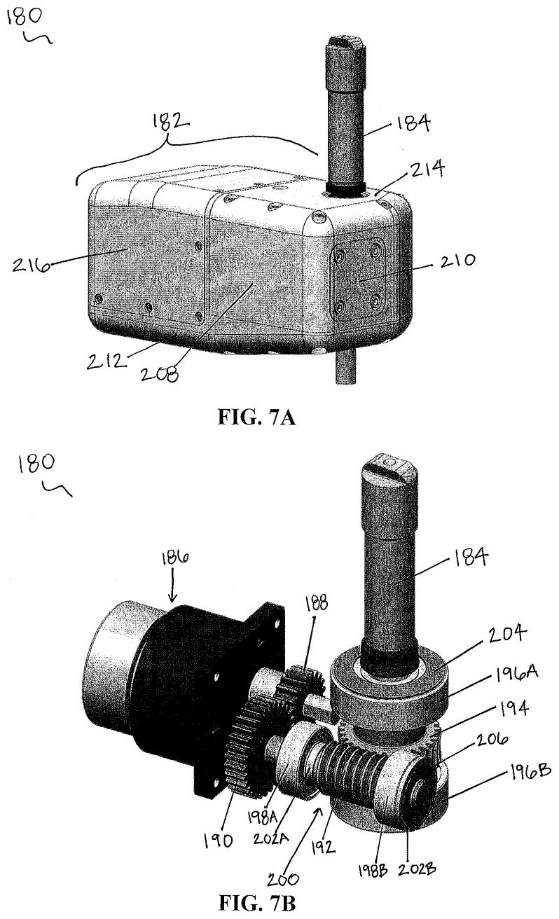

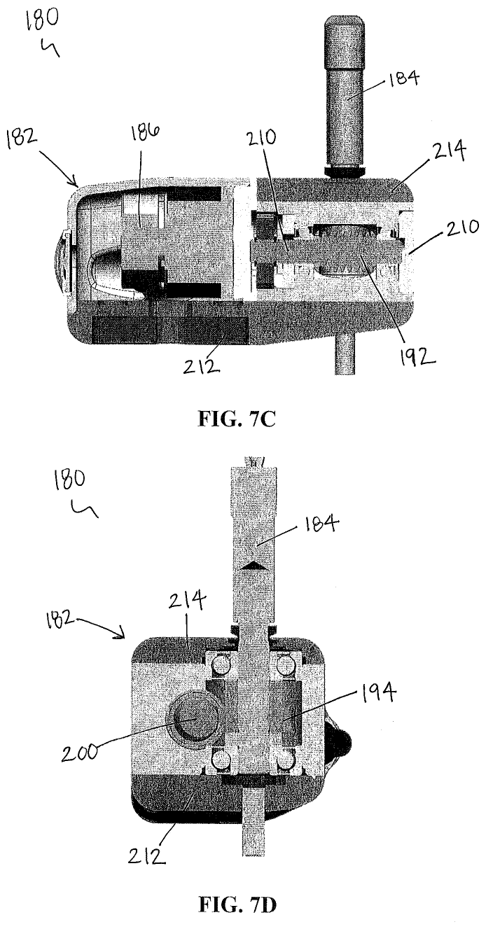

FIGS. 7A-7D depict a yaw joint (similar to joint 62 as discussed above) 180, according to one embodiment. More specifically, FIG. 7A depicts a yaw mechanism structure 182 having an output shaft 184 rotatably extending from the structure 182 such that the yaw joint 180 originates from the output shaft 184. The structure 182 in one embodiment can have a motor housing (or section) 216 and a drivetrain housing (or section) 208. Alternatively, the structure 182 has only one section or housing. The output shaft 184 can be rotated by the yaw mechanism structure 182 to cause the gross positioning robot (not shown) to rotate in relation to the robot support arm (not shown).

In one embodiment, as best shown in FIGS. 7B-7D, the structure 182 has a motor 184 rotatably coupled to the output shaft 184 via a series of gears such that the motor 184 can rotate the shaft 184. As best shown in FIG. 7B in which the housing of the structure 182 is not depicted, the gears in this exemplary implementation include a drive gear 188 rotationally constrained to the motor 186 and rotatably coupled to the driven gear 190. The driven gear 190 is rotationally constrained to a worm screw 192, which is threadably coupled to a worm wheel 194 such that rotation of the driven gear 190 causes rotation of the worm screw 192 and thus rotation of the worm wheel 194. The worm wheel 194 is rotationally constrained to the output shaft 184. As such, actuation of the motor 186 causes rotation of the drive gear 188, which rotates the driven gear 190, which rotates the worm screw 192, which rotates the worm wheel 194, which causes rotation of the output shaft 184. In one implementation, the gears can provide a total reduction that converts the high-speed motor output to the low speed and high torque required. Alternatively, other known gears or rotational elements may be used to cause the rotation of the output shaft 184.

The motor and drivetrain components can be supported and positioned within the structure 182 via various known mechanisms and features. Thus, the specific bearings, washers, spacers, and other components discussed below are exemplary and non-limiting. For example, in this embodiment, the output shaft 184 can be supported by two bearings 196A, 196B (e.g., opposing angular contact bearings) that can be flanged or capped to support the weight of the system. In addition, a retaining ring (not shown) can be provided that constrains the shaft axially against the bearings 196A, 196B. Torque can be transmitted from the worm gear 192 to the wheel 194 to the output shaft 184, for example with a key and keyway (not shown). The drive gear 188/driven gear 190 stage can protect the motor 186 from axial loads, while the intermediate parallel shaft 200 is supported against axial thrust on the worm with angular contact bearings 198A, 198B. The preload can be achieved with a disc spring 206 as shown, but other methods include the use of an axial wave or spring washers (not shown). Precision spacers 202 can be used to locate all bearing and gears on the shafts.

The gear train in one embodiment is disposed within the drivetrain housing 208. The worm screw bearing caps 210 retain the worm screw 192, the angular contact bearings 198, the spacers 202, and the disc spring 206 within the housing 208. As best shown in FIGS. 7C and 7D, the body 182 can have a baseplate 212 while the drivetrain housing 208 can have a top plate 214. Further, the motor 186, motor controller (not shown), and cabling (not shown) can be housed together within the motor housing 216. Further, the yaw mechanism structure (or “housing”) 182 can be securely fastened to the pitch mechanism structure (not shown) using a pair of locating pins (not shown) and low-profile shoulder screws (not shown) or the like to better transmit torque and move the entire device.

The specific yaw mechanism structure 182 is only one example of an appropriate structure with appropriate internal components that can be used to create the desired yaw movement. Any other known structure and known internal mechanisms can be incorporated herein to accomplish the same movement.

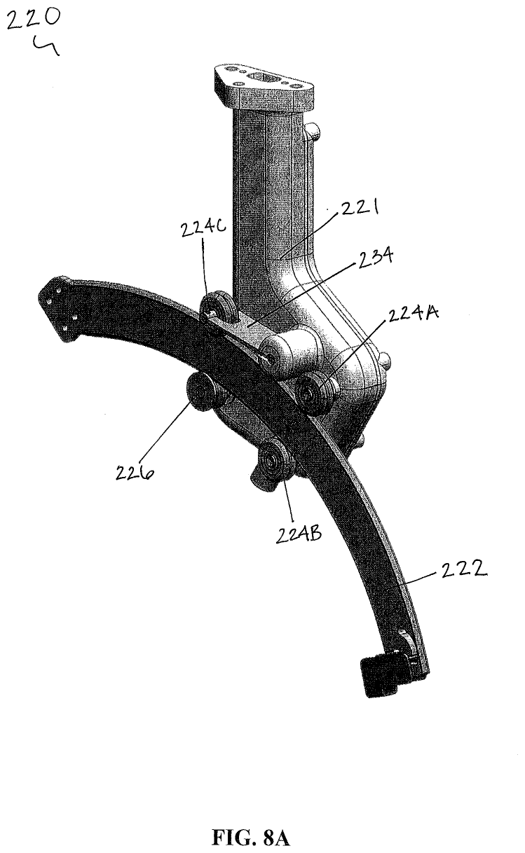

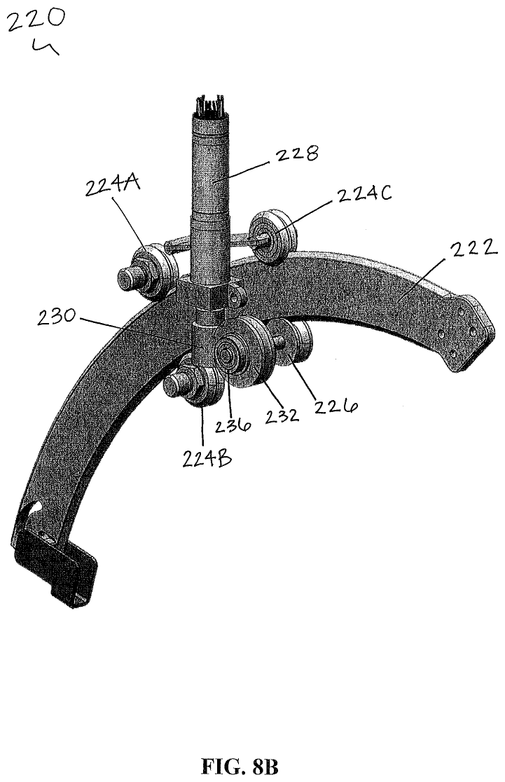

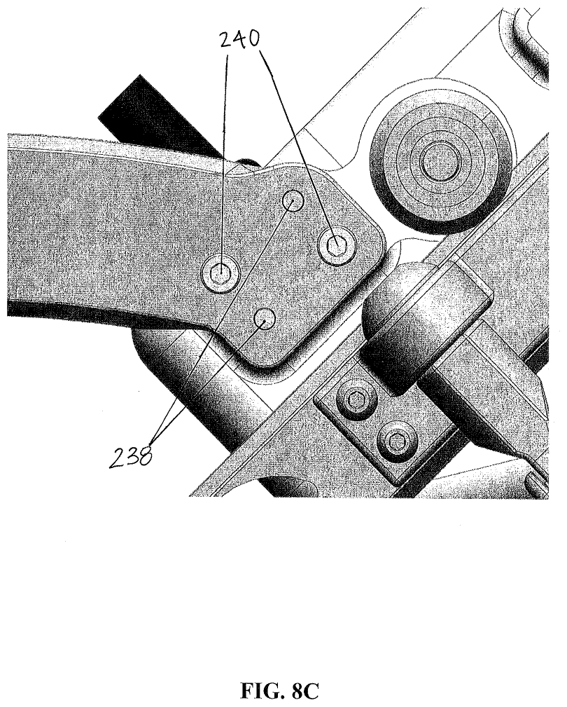

FIGS. 8A-8C depict a pitch mechanism 220 (similar to joint 64 as discussed above), according to one embodiment. The mechanism 220 has a body 221 and a curved output rail 222 slidably coupled to the body 221 such that the pitch results from movement of the output rail 222 in relation to the body 221. The output rail 222 is an elongate curved structure 222 that, according to one embodiment, constitutes of segment of a circular arc. Further, the output rail 222 is slidably coupled to the pitch mechanism body 221 as described below and further is fixedly coupled at one end of the rail 222 to the plunge housing (as discussed elsewhere herein) such that movement of the rail 222 causes rotation of the plunge housing (and any attached robotic surgical device) about the RCM (not shown). The rail 222 can be a bar or any other elongate curved structure.

In one embodiment, the output rail 222 is coupled to the pitch mechanism body 221 via rotatable bearings 224 and a drive roller 226. In the specific implementation as shown, there are three bearings 224 (e.g., grooved rotatable bearings) and the drive roller 226 (e.g., a grooved friction-drive roller) that are positioned on either side of the rail 222 such that the rail 222 is in contact with each of the bearings 224 and the roller 226 and can be urged to move translationally by the drive roller 226 in relation to the bearings 224. Further, as described in additional detail below, two of the bearings 224A, 224B are positioned opposite each other, above and below the pitch rail 222. Additionally, a third bearing 224C is mounted on the end of a leaf spring 234 tensionably coupled to the body 221 such that the third bearing 224C is positioned above the rail 222 opposite of the drive roller 226. The leaf spring 234 may be deflected when the rail 222 is installed, which may result in a perpendicular preload force being applied to the roller 226 through the rail 222 as a result of the leaf spring 234. The friction between the roller 226 and the pitch rail 222 from this preload force engages the roller 226 with the pitch rail 222 (e.g., the edge of the rail 226) without slipping. Alternatively, any tensionable component or other mechanism can be used to apply sufficient force to the rail 226 or the drive roller 226 to ensure that the roller 226 engages the rail 222 without any slipping. Further, instead of the specific configuration of bearings 224A-224C and drive roller 226, any one or more known components that can allow for movement of the rail 222 in relation to the body 221 can be incorporated herein to accomplish the desired pitch movement.

As best shown in FIG. 8B (in which the body 221 housing is not shown), the drive roller 226 is actuated by a motor 228 that is rotatably coupled to the drive roller 226 and thus the output rail 222 via a series of rotational elements. It is understood that the specific rotational elements described with respect to this specific embodiment are exemplary, and any known rotational elements or other mechanisms and configurations thereof can be used. The rotational elements in this exemplary implementation include a drive gear 230 that is a worm screw 230 that is rotationally constrained to the motor 228 and threadably/rotatably coupled to a worm wheel 232. Alternatively, the drive gear 230 can be any type of gear or component coupled to any type of wheel 232 or similar component. The worm wheel 232 is rotationally constrained to the roller 226, which is rotatably coupled to the pitch rail 222 as described above. Thus, actuation of the motor 228 causes rotation of the drive gear 230, which causes rotation of the worm wheel 232, which causes rotation of the roller 226 and thus translation of the rail 222. In one implementation, the gears can provide a total reduction that converts the high-speed motor output to the low speed and high torque required.

In one embodiment, the shaft containing the worm wheel 232 and the rotatable roller 226 can be supported with two bearings 236 on opposing ends of the shaft, which are on opposing sides of the worm wheel 232. Alternatively, the supporting components can be any known components or mechanisms for supporting a set of gears in a drivetrain. The shaft may be disposed within the body 221. Further, in one embodiment, the motor 228, gear train, motor controller (not shown), and cabling (not shown) are also housed together in the body 221. Further, as discussed in detail below, the output rail 222 is fastened to the plunge housing with alignment pins 238 and screws 240, as best shown in FIG. 8C. Alternatively, the output rail 222 can be coupled to the plunge housing via any known coupling mechanisms or features.

FIGS. 9A-9B depict a plunge device or mechanism (similar to joint 66 as discussed above) 250, according to one embodiment. More specifically, FIG. 9A depicts a plunge mechanism structure (or “body” or “housing”) 252 having an output rail 254 movably coupled to the structure 252 such that the plunge results from translational movement of the output rail 254 in relation to the structure 252 as a result of the structure 252 actuating the rail 254 to move. The output rail 254 is a substantially straight elongate structure 254 that is movably coupled to the plunge mechanism housing 252 as described below and further is fixedly coupled at one end of the rail 254 to the robot attachment clamp 256 such that movement of the rail 254 causes movement of any robotic device (not shown) disposed within the clamp 256, thereby translating the robotic device in and out of the port (or incision or opening) at the surgical site. The rail 254 can be a bar or any other elongate structure.

In one embodiment as best shown in FIG. 9B (in which the body 252 housing is not depicted), the output rail 254 is coupled to the plunge mechanism structure 252 via rotatable bearings 258A, 258B, 258C (e.g., three grooved bearings) and a drive roller 260 (e.g., a grooved friction drive roller). In the specific implementation as shown, the bearings 258A-C are positioned on either side of the rail 254 such that the rail 254 is in contact with each of the bearings 258A-C and can move translationally in relation to the bearings 258A-C. Two of the bearings 258A, 258B are positioned opposite each other on either side of the rail 254 at one end of the plunge mechanism structure 252. A third bearing 258C is coupled to a leaf spring 268 tensionably coupled to the body 221 opposite the roller 260 at a second end of the plunge mechanism structure 252. In some cases, a deflection in the leaf spring 268 may occur when the plunge rail 254 is installed, which may apply a preload force through the rail 254 perpendicularly against the roller 260 as a result of the leaf spring 268. The preload force and the friction created between the drive roller 260 and plunge rail 254 may enable the drive roller 260 to engage with the plunge rail 254 (e.g., the edge of the rail 254) without slipping. Alternatively, any tensionable component or other mechanism can be used to apply sufficient force to the rail 254 or the drive roller 260 to ensure that the roller 260 engages the rail 254 without any slipping. Further, instead of the specific configuration of bearings 258A-258C and drive roller 260, any one or more known components that can allow for movement of the rail 254 in relation to the body 252 can be incorporated herein to accomplish the desired plunge movement.

As best shown in FIG. 9B, according to one implementation, the drive roller 260 is actuated by a motor 262 that is rotatably coupled to the drive roller 260 and thus the output rail 254 via a series of rotational elements. It is understood that the specific rotational elements described with respect to this specific embodiment are exemplary, and any known rotational elements or other mechanisms and configurations thereof can be used. The rotational elements in this exemplary implementation include a drive gear 264 that is a worm screw 264 that is rotationally constrained to the motor 262 (e.g., at the output shaft of the motor 262) and threadably/rotatably coupled to a worm wheel 266. The worm wheel 266 is rotationally constrained to the roller 260, which is coupled to the edge of the plunge rail 254 by friction. Thus, actuation of the motor 262 causes rotation of the drive gear 264, which causes rotation of the worm wheel 266, which causes rotation of the roller 260 and thus translation of the rail 254. Alternatively, the actuation of rail 254 could be also be accomplished using a spur gear to engage with a rack attached to the plunge rail 254. In certain implementations, the gears can provide a total reduction that converts the high-speed motor output to the low speed and high torque required.

According to one embodiment, the shaft containing the worm wheel 266 and the roller 260 can be supported with two bearings 270 on opposing ends of the shaft, which are on opposing sides of the worm wheel 266. Alternatively, the supporting components can be any known components or mechanisms for supporting a set of gears in a drivetrain. The shaft may be disposed within the plunge housing 252. Further, in one embodiment, the motor 262, gear train, motor controller (not shown), and cabling (not shown) are also housed together in the plunge housing 252.

It is understood that alternative versions of these three yaw, pitch, and plunge joints can use any known mechanisms other than friction drive rollers. For example, each of the joints could use gears or be directly driven by a motor. In further alternatives, motion along the rails may not use gears, but may instead simply drive one of the support rollers to produce motion along the rail. In addition, hydraulic, pneumatic, or cable drives could be used in other known designs to produce the desired output motion.

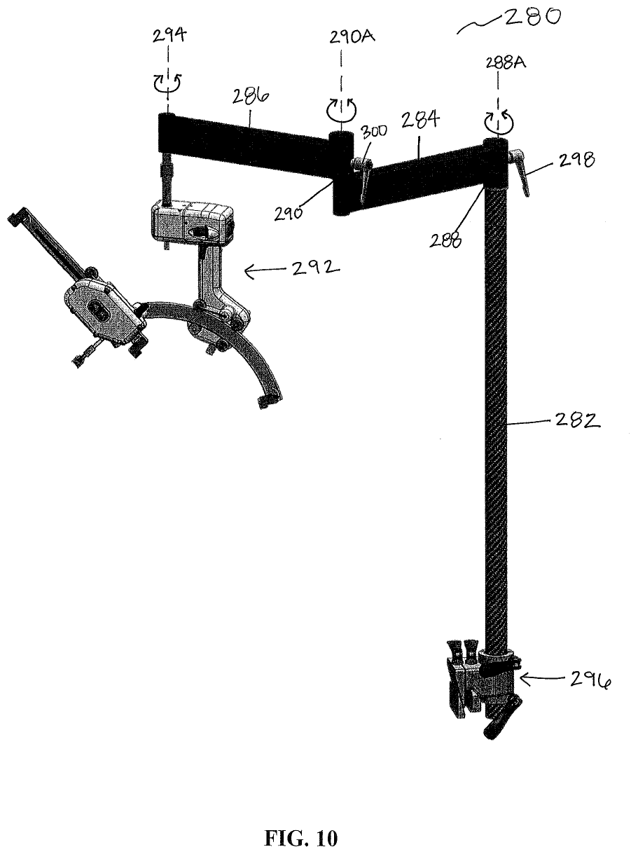

FIG. 10 depicts one embodiment of a robot support arm 280. In this implementation, the support arm 280 has a vertical (or “base column”) rod 282, a first elongate arm 284 rotatably coupled to the rod 282 at a first rotatable joint 288, and a second elongate arm 286 rotatably coupled to the first arm 284 at a second rotatable joint 290. The first joint 288 has a first axis of rotation 288A and the second joint 290 has a second axis of rotation 290A such that the two axes of rotation 288A, 290A are vertically parallel. This allows for planar (X/Y directions) positioning of the gross positioning robotic device 292 with respect to the patient. In addition, the yaw axis 294 of the gross positioning robotic device 292 (similar to yaw axis 62 as discussed above) is also vertically parallel to the other two axes of rotation 288A, 290A.

The vertical positioning (Z direction) of the support arm 280 can be adjusted at the bed rail (not shown) using the clamp 296. The vertical rod 282 may be coupled with the clamp 296 before or after the clamp is attached to the bed rail. Once a vertical placement of the rod 282 has been selected, the gross positioning robotic device 292 can be docked or otherwise attached to the support arm 280. Then, the arm 280 can be horizontally positioned as needed, including throughout the robotic surgical device (not shown) insertion process. Once a final position for the robotic surgical device (not shown) has been selected, the gross positioning robotic device 292 is docked with the robotic surgical device (not shown). Typically, this is accomplished by locating the RCM approximately at the port/incision/opening. At this point, the support arm 280 can be locked into position using joint locks 298, 300.

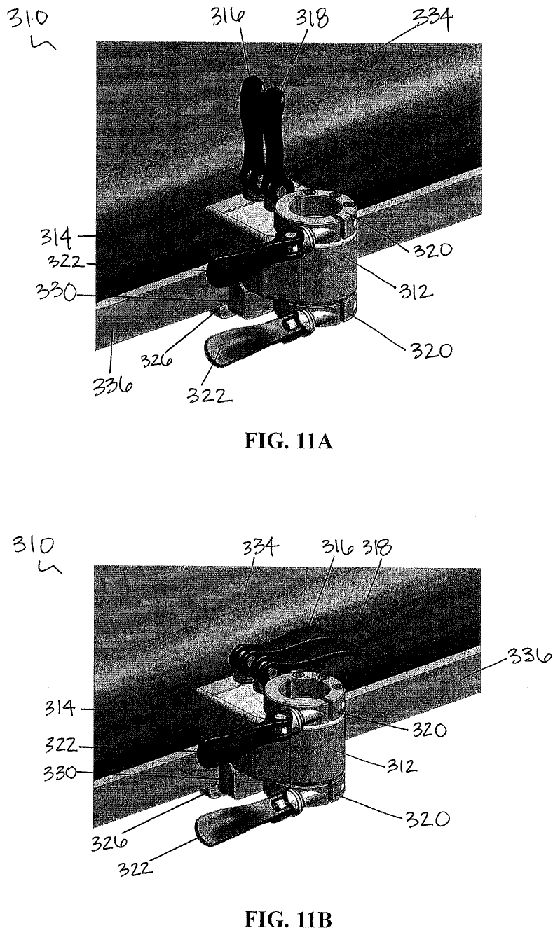

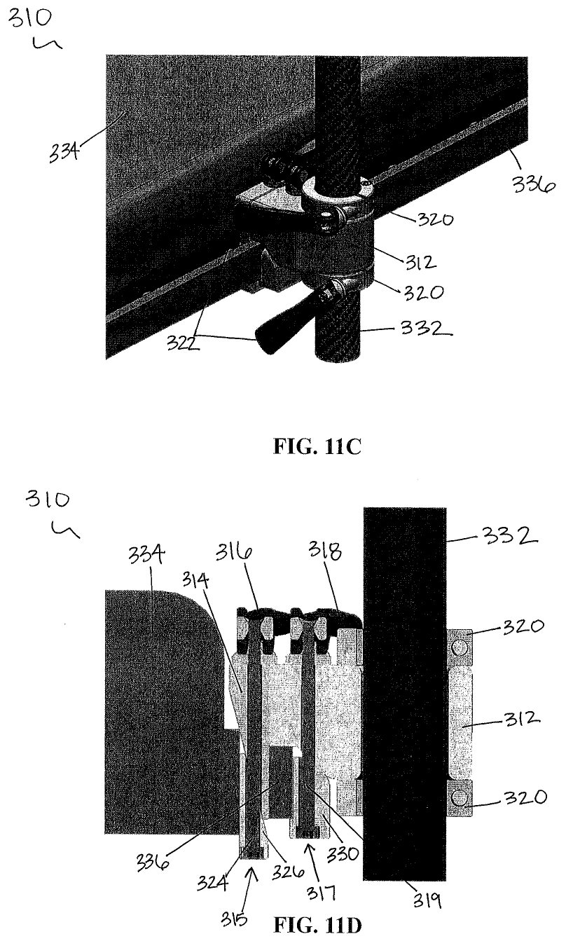

In one embodiment, a bed rail clamp 310 (similar to clamp 296, for example) is depicted in FIGS. 11A-11D. Loose or flexible bed rails have the potential to cause large deflections when using bed-mounted support arms, which act as cantilevered beams. To combat this, a robot support arm (such as arm 280) can be fastened to a standard surgical bed rail using the bed rail clamp 310. The clamp 310 has a clamp body 312 with two clamping shaft collars 320 coupled thereto, with one collar 320 on each end of the body 312, such that the clamp body 312 can receive the rod 332 of the support arm (similar to arm 280) and the collars 320 can secure the rod 332 thereto, as will be described in detail below. Further, as best shown in FIG. 11D, the clamp 310 can be securably attached to the bed rail 336 via the two attachment mechanisms 315, 317 attached to the clamp 310. The first or outer attachment mechanism 315 has an actuable bolt 324 that is actuated by the first handle 316 and is operably coupled to first and second wedges 314, 326. The first attachment mechanism 315 can be disposed between the rail 336 and the bed 334. The second or inner attachment mechanism 317 has an actuable bolt 319 that is actuated by the second handle 318 and is operably coupled to a clamping jaw 330. The second attachment mechanism 317 is disposed adjacent to the rail 336 on the outer face of the rail (opposite the first attachment mechanism 315.

In use, to secure the bed rail clamp 310 to the bed rail 336, the bed rail clamp 310 is positioned in relation to the rail 336 such that first attachment mechanism 315 is positioned between the rail 336 and the bed 334 while the second attachment mechanism 317 is positioned adjacent to the outer face of the rail 336. Once seated in the desired location, the handle 316 (e.g., a spreading clamp cam handle) can be urged into a down, locked position (as best shown in FIGS. 11B and 11D) to urge the actuable bolt 324 upward, thereby causing the bottom wedge 326 to be urged upward such that the angled face of the bottom wedge 326 engages with the angled face of the top wedge 314 such that the both wedges 326, 314 are urged laterally. As such, the bottom wedge 326 is urged against the bed 334 while the top wedge 314 is urged against the inner face of the rail 336, thereby tensionably securing the clamp 310 to the rail 336 and bed 334. After the handle 316 is in the locked position, the handle 318 (e.g., a rail clamp cam handle) can be urged into a down, locked position (as best shown in FIGS. 11B and 11D) to urge the actuable bolt 319 upward, thereby causing the clamp jaw 330 to move upward into contact with a bottom face of the rail 336, thereby securing the clamp 310 from shifting caused by forces applied to the vertical rod 332.

Once the bed rail clamp 310 is securely mounted to the bed 334, the rod 332 can be inserted through the clamping shaft collars 320 attached to the bed rail mount main body 312. As shown in FIG. 11C, the column clamp cam handles 322 are engaged in a locked position to secure the support column at the desired height by tightening the collars 320 around the rod 332. These column clamp cam handles 322 can be disengaged and re-engaged to allow the user to manually raise and lower the support arm column 332 as desired. It is understood that alternative methods for raising and lowering the support arm column can be used, such as the use of a hand crank or motor to adjust the height of the column via a leadscrew mechanism.

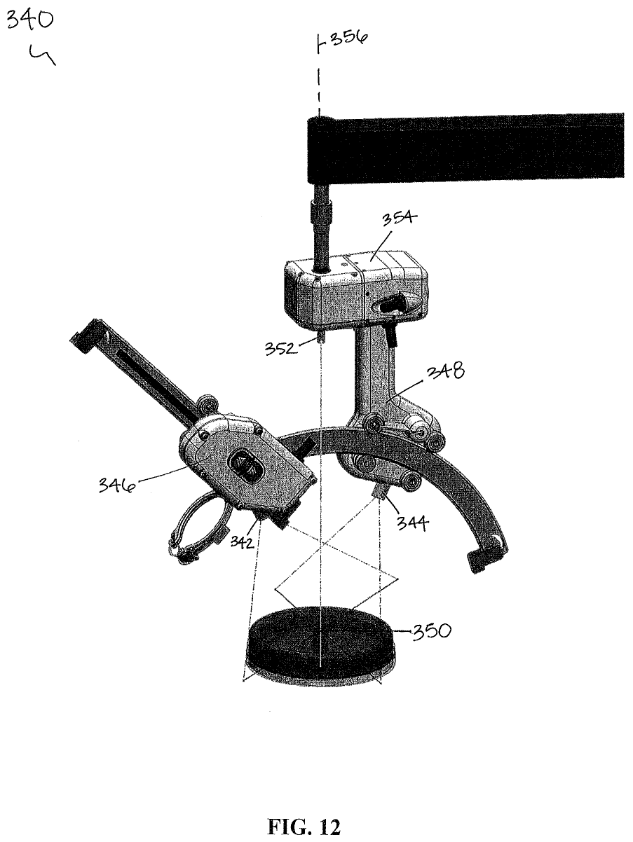

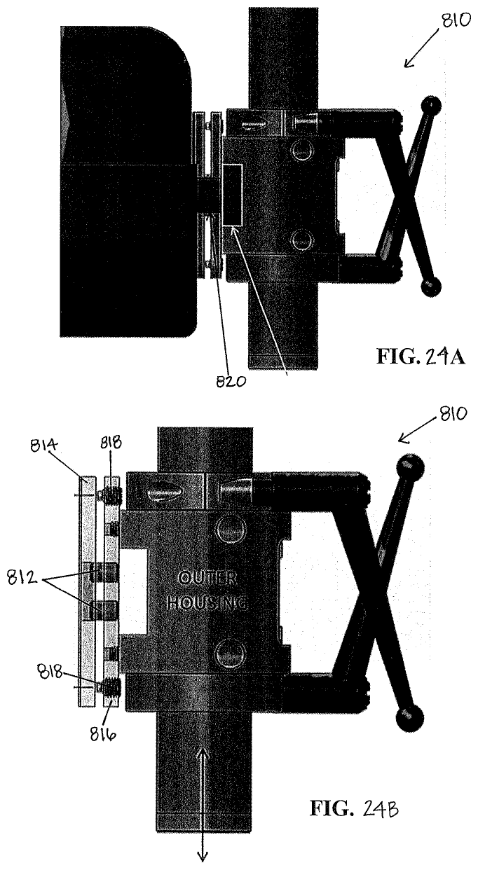

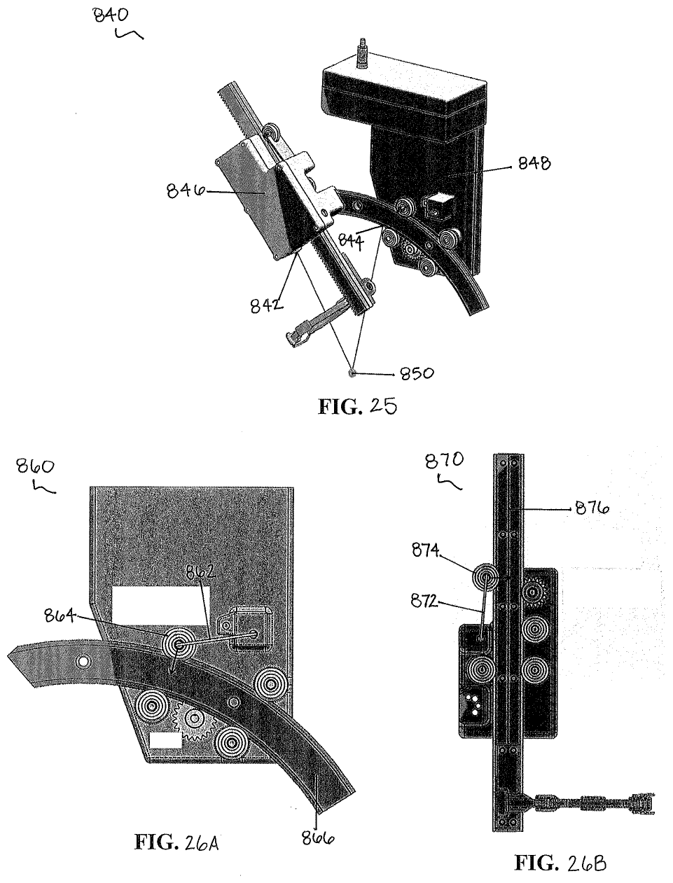

According to one alternative embodiment, any of the gross positioning robotic device embodiments herein can have an additional feature—laser-aided positioning. More specifically, one gross positioning robotic device 340 embodiment as shown in FIG. 12 has three line lasers 342, 344, 352 with one laser 342 disposed on the plunge housing 346, one laser 344 disposed on the pitch housing 348, and one laser 352 disposed on the yaw housing 354. The laser 352 may be mounted coaxially with the yaw axis 356. The lasers 342, 344, 352 are positioned and aimed to cause the laser light from each laser 342, 344, 352 to intersect at the RCM 350. Thus, in one embodiment, the lasers 342, 344, 352 can help with easy docking and positioning of the gross positioning robotic device 340, and the RCM 350 as shown by the lasers can easily be located at the patient incision/port/opening by the user. It is understood that alternative numbers of lasers may be used (such as, for example, two lasers as shown in FIG. 25). The various laser embodiments disclosed or contemplated herein can be incorporated into any gross positioning device embodiment disclosed herein.

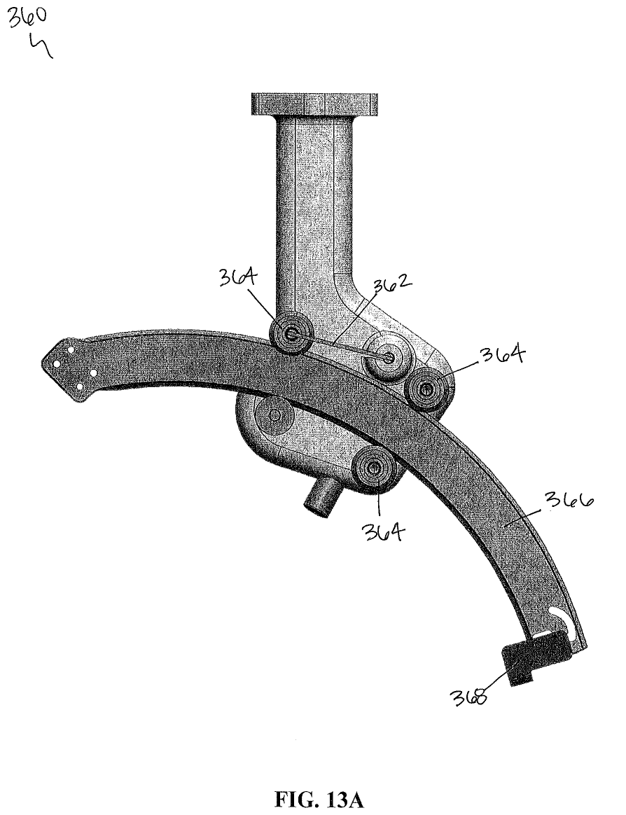

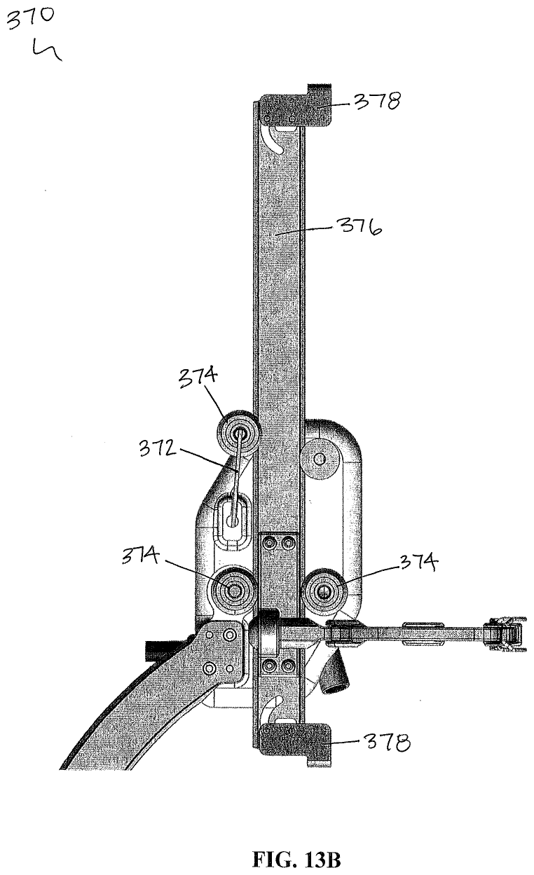

As shown in FIGS. 13A and 13B, and as discussed above with respect to FIGS. 8A and 9B, various implementations of the gross positioning device embodiments herein can also include tensioned mechanisms that provide tensioned force applied to at least one bearing of the pitch housing and/or the plunge housing to ensure contact of the bearings with the rails. More specifically, as shown in FIG. 13A, one exemplary embodiment of a pitch housing 360 has a leaf spring 362 that applies force to the bearing 364 that urges the bearing 364 into contact with the rail 366.

The leaf spring 362 can be manually de-tensioned or otherwise urged away from the rail 366, removing the bearing 364 from contacting the rail 366. This allows the rail 366 to be disengaged from the pitch housing 360. Additionally or alternatively, the pitch rail 376 includes a hard stop or “protrusion” 368. The toggleable protrusion 368 located at the end of the pitch rail 366 can be disengaged to allow for the rail 366 to be disengaged from the pitch housing 360. Then each subcomponent is easily disassembled as needed for cleaning and sterilization.

Similarly, as shown in FIG. 13B, one exemplary embodiment of a plunge housing 370 has a leaf spring 372 that applies force to the bearing 374 that urges the bearing 374 into contact with the rail 376. The leaf spring 372 can be manually de-tensioned or otherwise urged away from the rail 376, removing the bearing 374 from contacting the rail 376. This allows the rail 376 to be disengaged from the pitch housing 370. Additionally or alternatively, the plunge rail 376 includes a hard stop or “protrusion” 378. The toggleable protrusion 378 can be disengaged to allow the plunge rail 376 to be disengaged from the pitch housing 370. Then each subcomponent is easily disassembled as needed for cleaning and sterilization. It is understood that any known tensioning mechanism can be used in place of the leaf springs 362, 372 and any known toggleable mechanisms can be used in place of the protrusions 368, 378.

In a further alternative embodiment, any gross positioning robotic device as disclosed or contemplated herein can be controlled at the bedside using a local interface, such as a button (e.g., actuators 88, 90, 92) a joystick (not shown), a tablet, or any other known interface to drive each joint independently. The user can jog each joint individually or simultaneously with the interface. The gross positioning robotic device can be set aside while the robotic surgical device is inserted and then easily be introduced for docking when needed with this function. The interface can be intuitive, with the button or joystick articulation direction corresponding to the drive direction. To achieve this, the user interface can be localized at each joint or can be centrally located. For robot extraction, the robotic surgical device can be un-docked, and the gross positioning robotic device can be jogged out of the way.

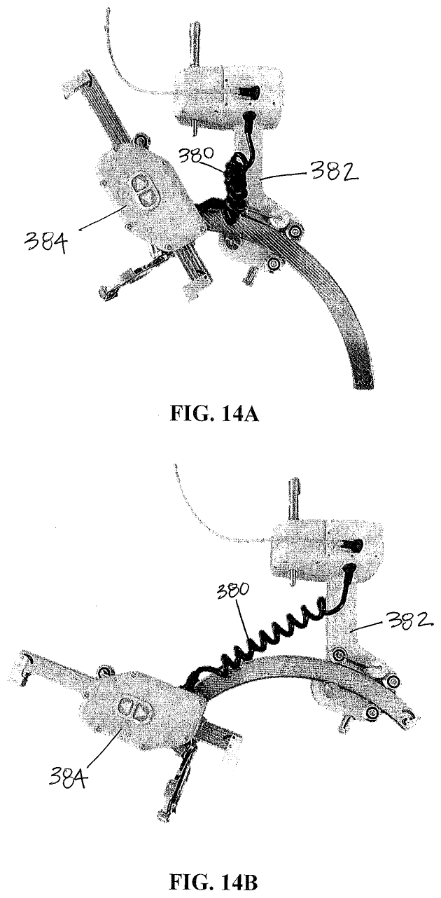

FIGS. 14A and 14B depict another alternative feature of any gross positioning robotic device disclosed or contemplated herein: a coiled cable 380. In this embodiment, the cable 380 couples the pitch housing 382 to the plunge housing 384 as shown. The cable 380 is coiled to allow for the full range of pitch angles without cable entanglement by naturally retracting and extending as the pitch joint is pitched. In alternative designs, a slip ring or joint capsule could be used on any or all the joints. These designs could have continuous or very large ranges of motion.

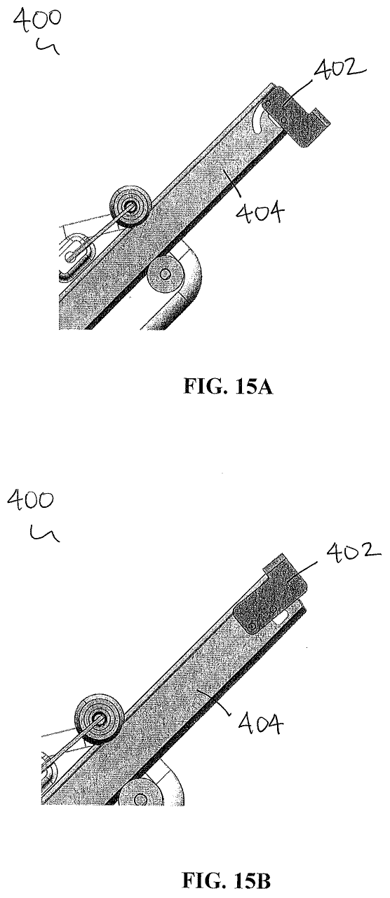

FIGS. 15A and 15B depict another alternative feature of any rail on any gross positioning robotic device disclosed or contemplated herein: a toggleable hard stop or “protrusion” 402. As shown, the hard stop 400 is positioned on the pitch output rail 404. The toggleable hard stop 402 is located at the end of the pitch rail 404 and can be disengaged to allow for the rail 404 to be disengaged from the pitch housing. FIG. 15A shows the hard stop 402 in an engaged position such that the rail 404 cannot be disengaged from the pitch housing. FIG. 15B shows the hard stop 402 toggled from FIG. 15A, and the hard stop 402 is in a disengaged position such that the rail 404 can be disengaged from the pitch housing It is understood that a hard stop can also be positioned on a plunge output rail as shown above in FIG. 13A.

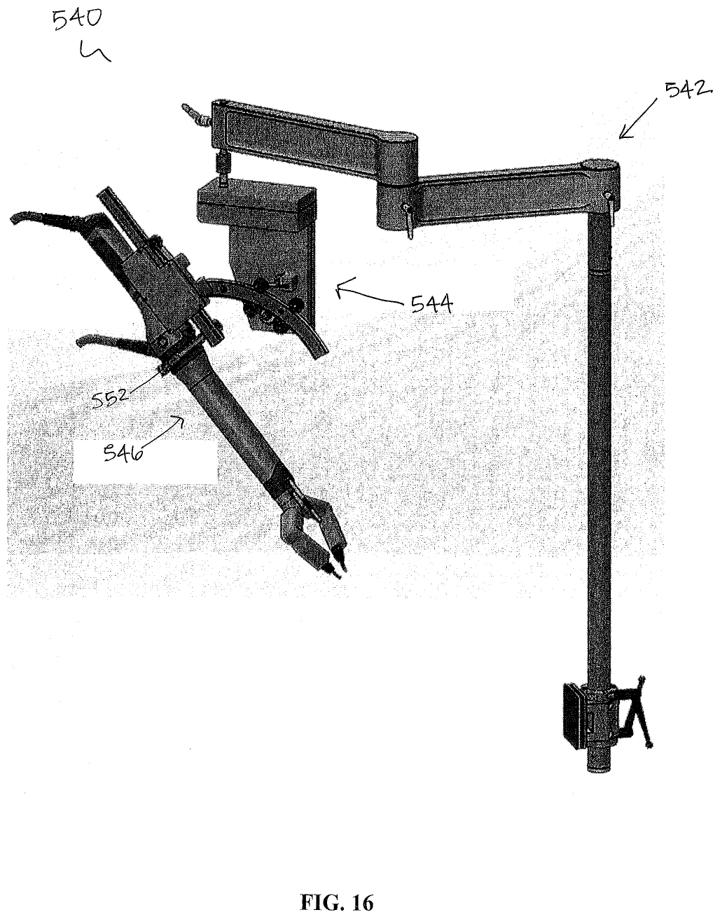

Another embodiment of a robotic surgical device positioning system 540 is depicted in FIG. 16. It is understood that the various embodiments as disclosed in FIGS. 16-23 are substantially similar to the device implementations disclosed or contemplated above in FIGS. 2A-15B, with substantially similar components, features, and functions, except as expressly discussed herein.

The system 540 in FIG. 16 includes a passive support arm 542 and a gross positioning robotic device 544 rotatably coupled to the arm 542. Further, any known robotic device 546, which in this specific exemplary implementation is represented by the device 546 as shown, can be removably coupled to the device 544 such that the device 546 is disposed through an opening, orifice, incision, or port into the target cavity of the patient.

According to another embodiment of a gross positioning robotic device 560 as shown in FIGS. 17A and 17B, the device 560 has three joints: yaw (joint 1) 562, pitch (joint 2) 564, and plunge (joint 3) 566. Yaw 562 and pitch 564 rotate about the RCM, and the plunge 566 moves through the RCM 568. In this embodiment, each joint is fully decoupled and is controlled independently. Further, in certain embodiments, motors and motor controllers (not shown) are co-located at each joint 562, 564, 566. In addition, it is understood that each joint 562, 564, 566 can be, but does not have to be, backdriveable.

The yaw joint 562 originates from the body 570 of the gross positioning device 560, and more specifically in the yaw mechanism structure 572. More specifically, a rotatable yaw output shaft 574 extends from the yaw mechanism structure 572 and constitutes the yaw joint 562. As such, rotation of the output shaft 574 creates the yaw at the yaw joint 562.

The pitch joint 564 also originates from the body 570 of the device 560, and more specifically in the pitch mechanism structure 576. More specifically, an output rail 578 is operably coupled to the pitch mechanism structure 576 via rotatable bearings 580 such that movement of the output rail 578 in relation to the pitch mechanism structure 576 (as described in detail below) creates the pitch joint 564. As such, actuation of the output rail 578 creates the pitch at the pitch joint 564.

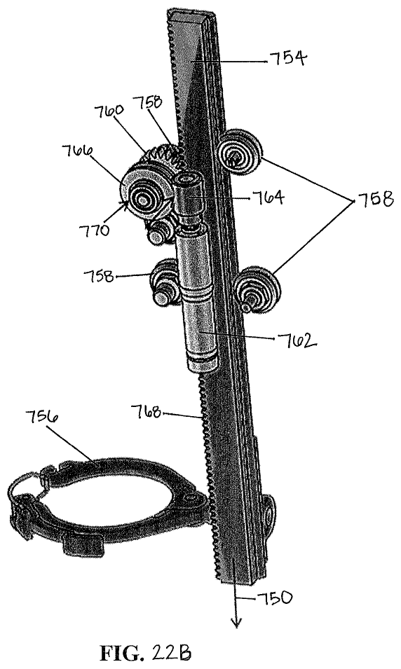

The plunge joint 566 originates from the plunge mechanism structure 582, which is operably coupled to the output rail 578. More specifically, an extendable rail 584 is operably coupled to the plunge mechanisms structure 582 via rotatable bearings 586 (as best shown according to one example in FIGS. 22A and 22B below). As such, extension of the extendable rail 584 creates the plunge at the plunge joint 566.

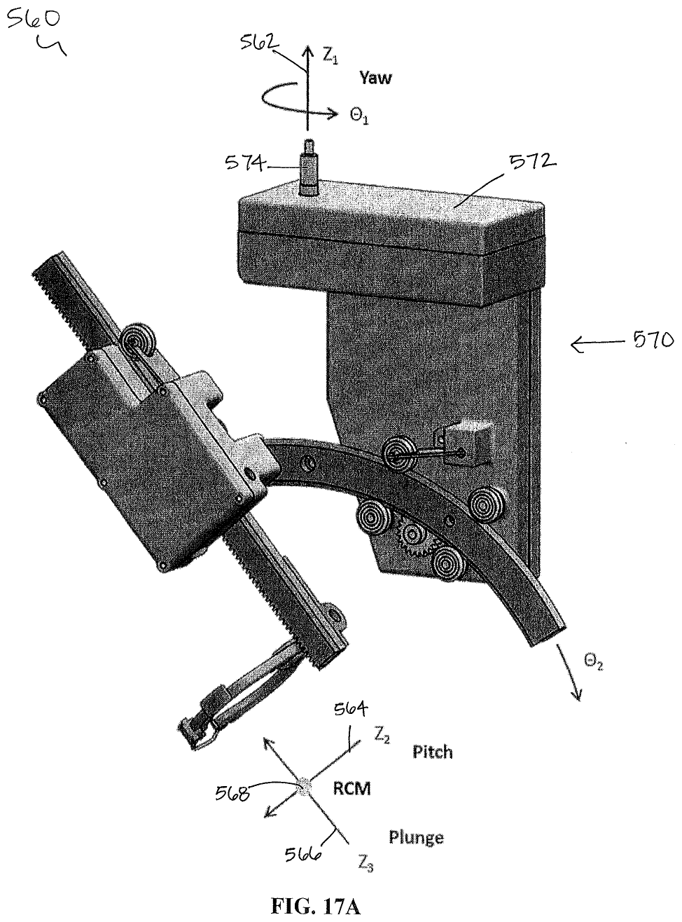

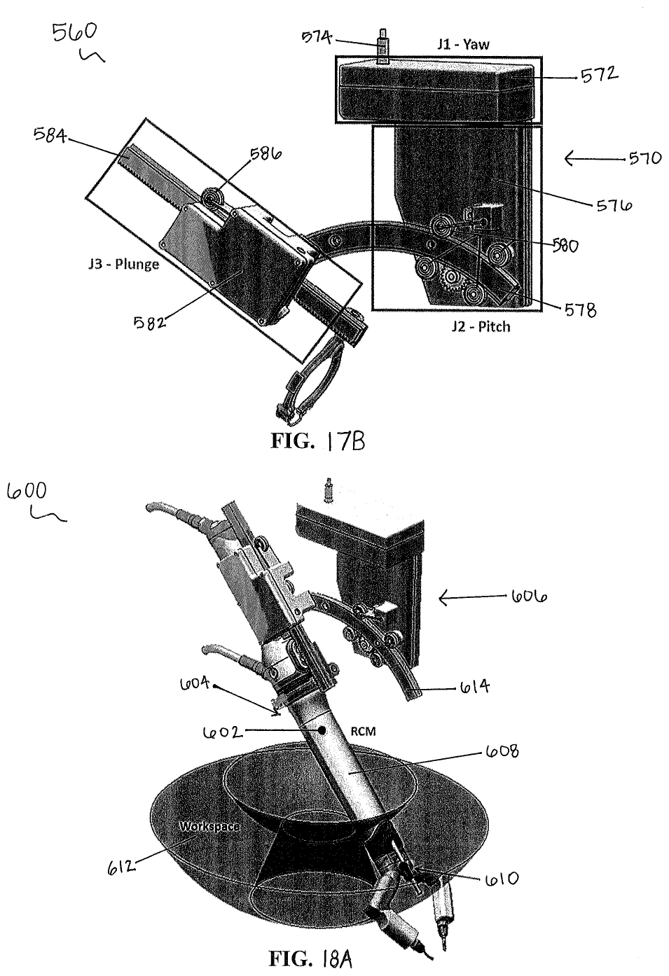

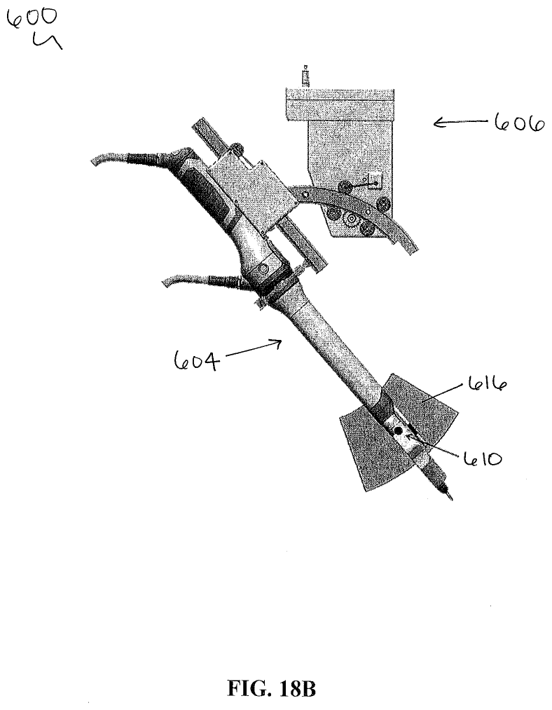

As best shown in FIGS. 18A and 18B, another implementation of a device positioning system 600 has an RCM 602 that is located at the center of the circular arced rail 614. Alternatively, the RCM 602 can be disposed in any known position in relation to the gross positioning device. When a robotic surgical device—such as device 604—is docked (or otherwise coupled) to the gross positioning robotic device 606, the RCM 602 is within the elongate body (or tube) 608 of the device 604 and approximately colinear with its kinematic origin 610. FIG. 18A also depicts the workspace 612 of the gross positioning device 606 in the context of the robotic surgical device 604. The kinematic origin 610 of the robotic device 604 is shown. The gross positioning device 606 can be used to move the kinematic origin 610 to any location within the toroidal workspace 612. The cross-section of the workspace 612, which is governed by pitch and plunge, is an annulus sector 616 as best shown in FIG. 18B.

Returning to FIG. 18A, the yaw joint (such as joint 562 as discussed in detail above with respect to FIG. 17A) is a rotational joint and can be articulated over a sweep angle of at least 165 degrees. In the embodiment of FIG. 18A, the workspace 612 depicts yaw with 360 degrees of travel, meaning it can rotate endlessly in either direction (as cabling adjustment permits). The pitch joint (such as joint 564 as discussed in detail above with respect to FIG. 17A) allows rotation about the RCM 602 with motion along the output rail 614. For pitch, according to certain implementations, a 40-degree arc can be traversed, with pitch angles approximately between 20 and 60 degrees from vertical. In addition, the plunge joint (such as joint 566 as discussed in detail above with respect to FIG. 17A) is a translational joint and can be translated a total length of at least 100 cm, according to various embodiments.

FIGS. 19A and 19B depict one exemplary embodiment of an output shaft 640 for a yaw joint. FIG. 19A depicts a perspective view of the shaft 640, while FIG. 19B shows an exploded cross-sectional view. As best shown in FIG. 19A, the output shaft 640 is coupled with the robot support arm 642. In this specific implementation, the output shaft 640 is a threaded D-shaft 640. The upper section 644 of the shaft 640 has a female connection opening 646 with threads 648 defined therein that are coupleable with the male connection protrusion 650 of the lower section 652 with threads 654 defined thereon. The coupled threads 648, 654 support the weight of the device(s). A shaft coupler 656 is disposed around the shaft 640 and has locking set screws 658A, 658B that allow the transmission of torque without unscrewing the two sections 644, 652 of the shaft 644. Alternatively, other known quick connection mechanisms can be used for easy docking and undocking of the gross positioning robotic device 660 to the robot support arm 642, such as for cleaning and sterilization.

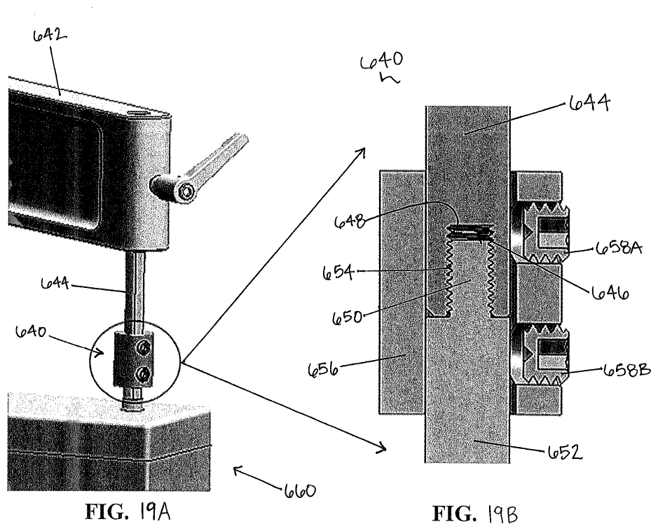

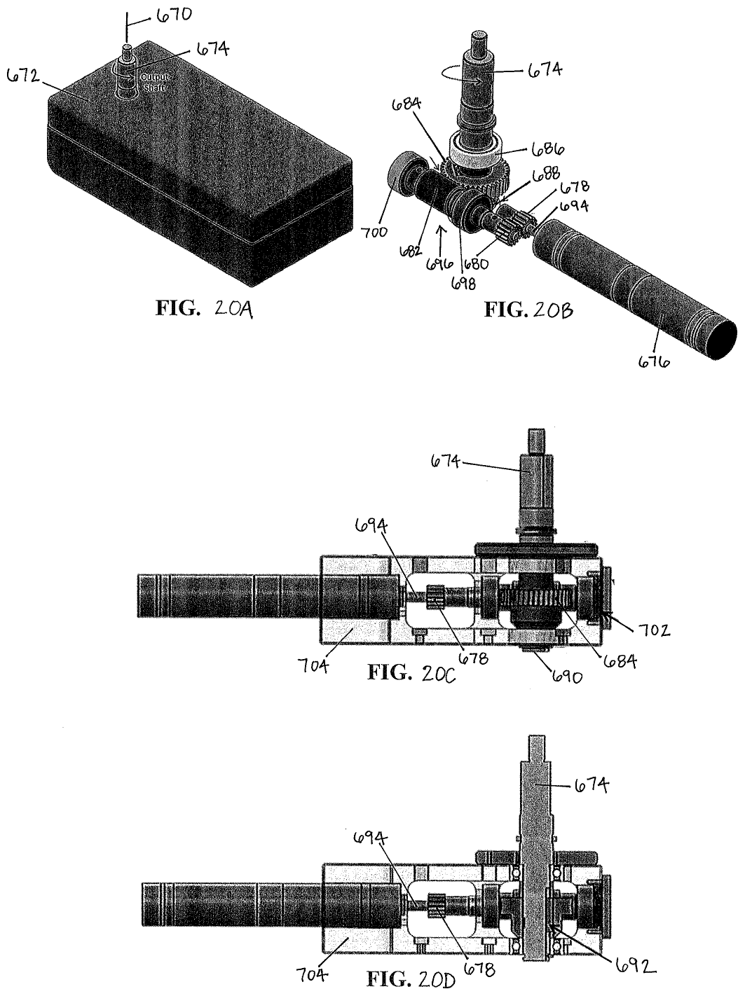

FIGS. 20A-20D depict the internal mechanisms of a yaw joint (such as joint 562 as discussed above) 670, according to one embodiment. More specifically, FIG. 20A depicts a yaw mechanism structure 672 having an output shaft 674 rotatably extending from the structure 672 such that the yaw joint 670 originates from the output shaft 674. The output shaft 674 rotates to cause the gross positioning robot (not shown) to rotate in relation to the robot support arm (not shown).

In one embodiment, as best shown in FIGS. 20B-20D, the output shaft 674 is actuated by a motor 676 that is rotatably coupled to the output shaft 674 via a series of rotational elements. The gears include a drive gear 678 rotationally constrained to the motor 676 and rotatably coupled to the driven gear 680. The driven gear 680 is rotationally constrained to a worm screw 682, which is threadably coupled to a worm wheel 684 such that rotation of the driven gear 680 causes rotation of the worm screw 682 and thus rotation of the worm wheel 684. In one implementation, the gears provide a total reduction of 5000:1, converting the high-speed motor output to the low speed and high torque required. The output shaft 674 is supported by two bearings 686, 688 that can be flanged or capped to support the weight of the system. In addition, a retaining ring 690 can be provided that constrains the shaft axially against the bearings 686, 688. Torque is transmitted from the worm gear 682, 684 to the output shaft 674 with a key and keyway 692. The drive gear 678/driven gear 680 stage protects the motor output shaft 694 from axial loads, while the intermediate parallel shaft 696 is supported against axial thrust on the worm with preloaded angular contact bearings 698, 700. The preload can be achieved with an axial wave spring 702 as shown, but other methods include the use of spring washers or disk springs (not shown). Spacers (not shown) can be used to locate all bearing and gears on the shafts. The gear train in one embodiment is disposed within a motor block 704. The assembled motor block 704, motor controller (not shown), and cabling (not shown) can be housed together. Further, the yaw mechanism structure (or “housing”) 672 can be securely fastened to the pitch mechanism structure (not shown) using shoulder bolts or the like to better transmit torque and move the entire device.

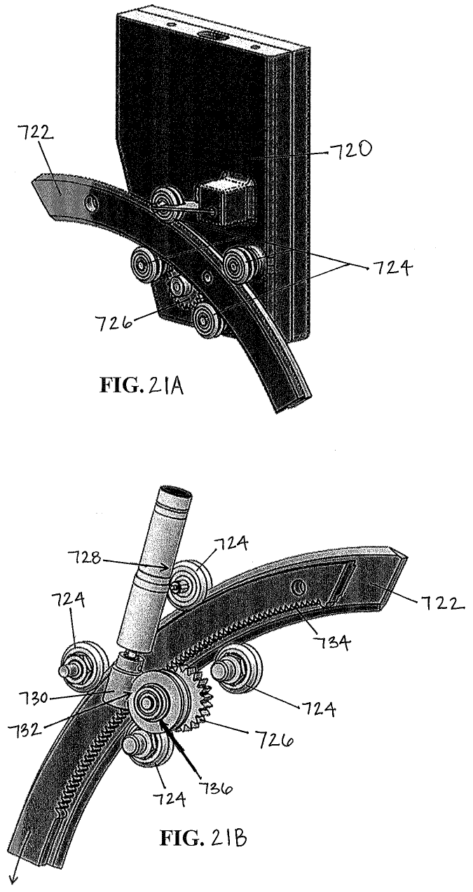

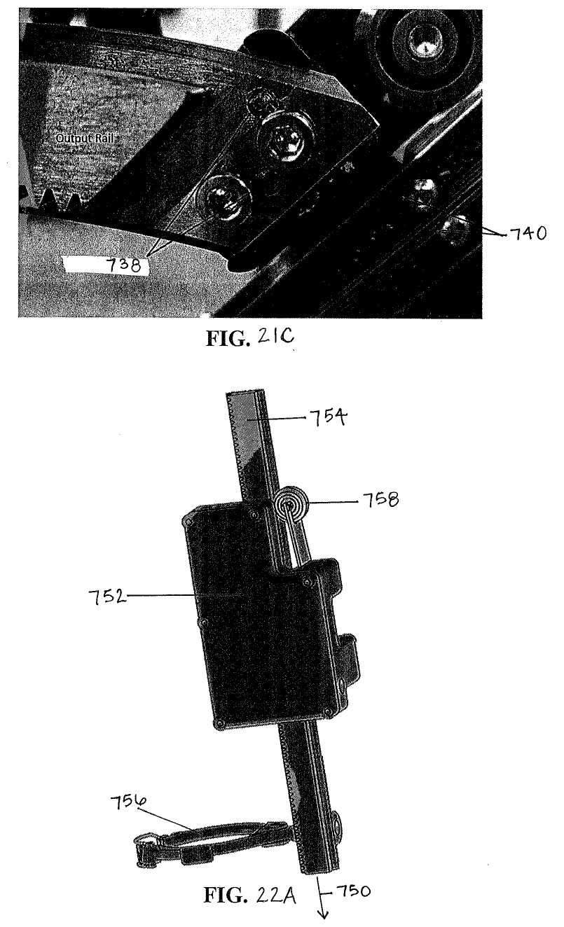

FIGS. 21A-21C depict the internal mechanisms of a pitch joint (such as joint 564 as discussed above), according to one embodiment. More specifically, FIG. 21A depicts a pitch mechanism structure (or “housing”) 720 having an output rail 722 slidably coupled to the structure 720 such that the pitch results from movement of the output rail 722 in relation to the structure 720. The output rail 722 is an elongate curved structure 722 that, according to one embodiment, constitutes of segment of a circular arc. Further, the output rail 722 is slidably coupled to the pitch mechanism housing 720 as described below and further is fixedly coupled at one end of the rail 722 to the plunge housing (not shown) such that movement of the rail 722 causes rotation of the plunge housing (and any attached robotic surgical device) about the RCM (not shown).

In one embodiment, the output rail 722 is coupled to the pitch mechanism structure 720 via rotatable bearings 724. In the specific implementation as shown, there are two pairs of bearings 724 that are positioned on either side of the rail 722 such that the rail 722 is in contact with each of the bearings 724 and can move translationally in relation to the bearings 724. Further, as described in additional detail below, the rail 722 is threadably coupled to a rotatable gear 726.

As best shown in FIG. 21B, the output rail 722 is actuated by a motor 728 that is rotatably coupled to the output rail 722 via a series of gears or other rotation elements. The gears include a drive gear 730 that is a worm screw 730 that is rotationally constrained to the motor 728 and threadably/rotatably coupled to a worm wheel 732. The worm wheel 732 is rotationally constrained to the rotatable gear 726, which is threadably coupled to the teeth 734 of the rail 722 such that rotation of the worm wheel 732 causes rotation of the rotatable gear 726 and thus translation of the teeth 734 (and thus the rail 722). In one implementation, the gears provide a total reduction of 12900:1, converting the high-speed motor output to the low speed and high torque required. The shaft containing the worm wheel 732 and the rotatable gear 726 is supported with two bearings 736 on opposing ends of the shaft. In one embodiment, the motor 728, gear train, motor controller (not shown), and cabling (not shown) are housed together in the pitch housing 720. Further, the output rail 722 is fastened to the plunge housing (discussed below) with alignment pins 738 and screws 740, as best shown in FIG. 21C.

FIGS. 22A-22B depict a plunge joint (such as joint 566 as discussed above) 750, according to one embodiment. More specifically, FIG. 22A depicts a plunge mechanism structure (or “housing”) 752 having an output rail 754 slidably coupled to the structure 752 such that the plunge or sliding movement results from translational movement of the output rail 754 in relation to the structure 752. The output rail 754 is a substantially straight elongate structure 754 that is slidably coupled to the plunge mechanism housing 752 as described below and further is fixedly coupled at one end of the rail 754 to the robot attachment clamp 756 such that movement of the rail 754 causes movement of any robotic device (not shown) disposed within the clamp 756, thereby translating the robotic device into and out of the port (or incision or opening) at the surgical site.

In one embodiment, the output rail 754 is coupled to the plunge mechanism structure 752 via rotatable bearings 758. In the specific implementation as shown, there are two pairs of bearings 758 that are positioned on either side of the rail 754 such that the rail 754 is in contact with each of the bearings 758 and can move translationally in relation to the bearings 758. Further, as described in additional detail below, the rail 754 is threadably coupled to a rotatable gear 760.

As best shown in FIG. 22B, the output rail 754 is actuated by a motor 762 that is rotatably coupled to the output rail 754 via a series of gears or other rotation elements. The gears include a drive gear 764, for example, a worm screw 764 or other rotatable element, that is rotationally constrained to the motor 762 and threadably/rotatably coupled to a worm wheel 766. The worm wheel 766 is rotationally constrained to the rotatable gear 760, which is threadably coupled to the teeth 768 of the rail 754 such that rotation of the worm wheel 766 causes rotation of the rotatable gear 760 and thus translation of the teeth 768 (and thus the rail 754). In one implementation, the gears provide a total reduction of 840:1, converting the high-speed motor output to the low speed and high torque required. The shaft containing the worm wheel 766 and the rotatable gear 760 is supported with two bearings 770 on opposing ends of the shaft. In one embodiment, the motor 762, gear train, motor controller (not shown), and cabling (not shown) are housed together in the plunge housing 752.

It is understood that alternative versions of these three joints can use any known mechanisms other than gears. For example, each of the joints could be directly driven by a motor. In further alternatives, motion along the rails may not use gears, but may instead simply drive one of the support rollers to produce motion along the rail. In addition, hydraulic, pneumatic, or cable drives could be used in other known designs to produce the desired output motion.

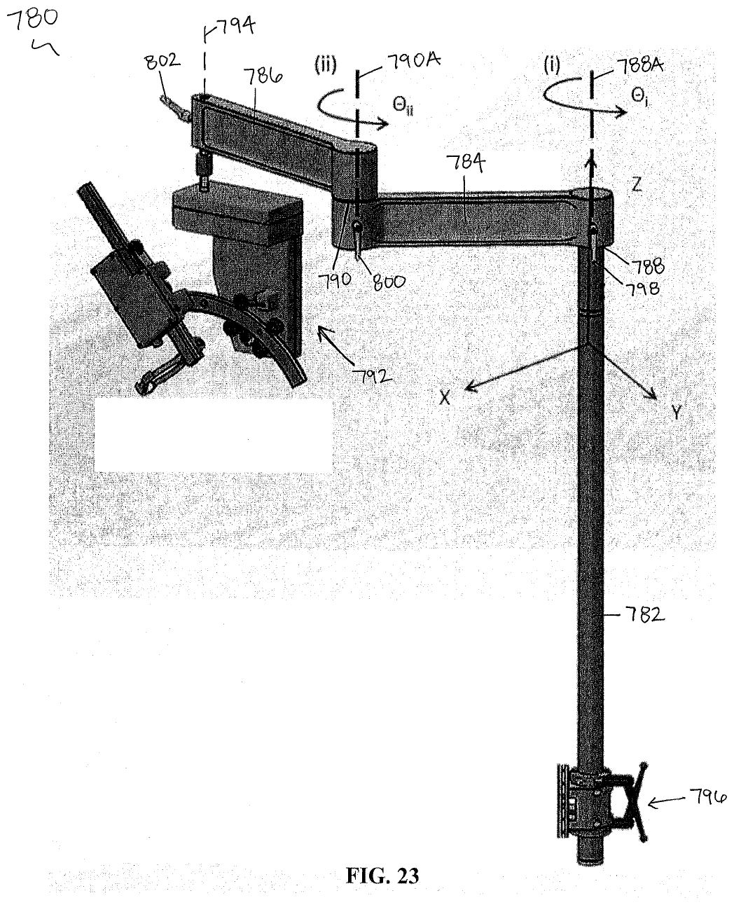

FIG. 23 depicts one embodiment of a robot support arm 780. In this implementation, the support arm 780 has a vertical (or “base”) rod 782, a first elongate arm 784 rotatably coupled to the rod 782 at a first rotatable joint 788, and a second elongate arm 786 rotatably coupled to the first arm 784 at a second rotatable joint 790. The first joint 788 has a first axis of rotation 788A, and the second joint 790 has a second axis of rotation 790A such that the two axes of rotation 788A, 790A are vertically parallel. This allows for planar (X/Y directions) positioning of the gross positioning robotic device 792 with respect to the patient. In addition, the yaw axis 794 of the gross positioning robotic device 792 is also vertically parallel to the other two axes of rotation 788A, 790A.

The vertical positioning (Z direction) of the support arm 780 can be adjusted at the bed rail (not shown) using the clamp 796. Once vertical placement has been selected, the gross positioning robotic device 792 can be docked or otherwise attached to the support arm 780. Then the arm 780 can be horizontally positioned as needed, including throughout the robotic surgical device (not shown) insertion process. Once a final position for the robotic surgical device (not shown) has been selected, the gross positioning robotic device 792 is docked with the robotic surgical device (not shown). Typically, this is accomplished by locating the RCM approximately at the port/incision/opening. At this point, the support arm 780 can be locked into position using joint locks 798, 800. Joint lock 802 is used to support the gross positioning robotic device 792 output shaft as noted above.

In one embodiment, a bed rail clamp 810 (similar to clamp 796) is depicted in FIGS. 24A-24B. Loose or flexible bed rails have the potential to cause large deflections when using bed-mounted support arms, which act as cantilevered beams. To combat this, a robot support arm (such as arm 780) can be fastened to a standard surgical bed rail using the spreading clamp 810. The clamp 810 has two alignment pins 812 that support the back plate 814 and act like linear bearings. The back plate 814 is pushed against the bedside, and the front plate 816 against the bed rail, using four set screws 818. This puts the bed rail mounting bolts 820 in tension, limiting any potential deflection. The outer housing rests on the bed rail and allows for height adjustment of the support arm (such as arm 780).

According to one alternative embodiment, any of the gross positioning robotic device embodiments herein can have an additional feature—laser-aided positioning. More specifically, the gross positioning robotic device 840 embodiment as shown in FIG. 25 has two line lasers 842, 844, with one laser 842 disposed on the plunge housing 846 and one laser 844 disposed on the pitch housing 848. The lasers 842, 844 are positioned and aimed to cause the laser light from each laser 842, 844 to intersect at the RCM 850. Thus, the lasers 842, 844 can help with easy docking and positioning of the gross positioning robotic device 840, and the RCM 850 as shown by the lasers can easily be located at the patient incision/port/opening by the user.

As shown in FIGS. 26A and 26B, various implementations of the gross positioning device embodiments herein can also include tensioned mechanisms that provide tensioned force applied to at least one bearing of the pitch housing and/or the plunge housing to ensure contact of the bearings with the rails. More specifically, as shown in FIG. 26A, one embodiment of a pitch housing 860 has a leaf spring 862 that applies force to the bearing 864 that urges the bearing 864 into contact with the rail 866. The leaf spring 862 can be manually de-tensioned, removing the bearing 864 from contacting the rail 866. This allows the rail 866 to be disengaged from the pitch housing 860. Then each subcomponent is easily disassembled as needed for cleaning and sterilization. Similarly, as shown in FIG. 26B, one embodiment of a plunge housing 870 has a leaf spring 872 that applies force to the bearing 874 that urges the bearing 874 into contact with the rail 876. The leaf spring 872 can be manually de-tensioned, removing the bearing 874 from contacting the rail 876. This allows the rail 876 to be disengaged from the pitch housing 870. Then each subcomponent is easily disassembled as needed for cleaning and sterilization. It is understood that any known tensioning mechanism can be used in place of the leaf springs.

In a further alternative embodiment, any gross positioning robotic device as disclosed or contemplated herein can be controlled at the bedside using a local interface, such as a button or a joystick (not shown), to drive each joint independently. The user can jog each joint individually or simultaneously with the interface. The gross positioning robotic device can be set aside while the robotic surgical device is inserted and then easily be introduced for docking when needed with this function. The interface can be intuitive, with the button or joystick articulation direction corresponding to the drive direction. To achieve this, the user interface can be localized at each joint or can be centrally located. For robot extraction, the robotic surgical device can be un-docked, and the gross positioning robotic device can be jogged out of the way.

While multiple embodiments are disclosed, still other embodiments will become apparent to those skilled in the art from the following detailed description, which shows and describes illustrative embodiments. As will be realized, the various implementations are capable of modifications in various obvious aspects, all without departing from the spirit and scope thereof. Accordingly, the drawings and detailed description are to be regarded as illustrative in nature and not restrictive.

Although the various embodiments have been described with reference to preferred implementations, persons skilled in the art will recognize that changes may be made in form and detail without departing from the spirit and scope thereof.

What is claimed is:

1. A gross positioning system for use with a robotic surgical device, the system comprising:
(a) a positioning body;
(b) a yaw mechanism operably coupled to the positioning body at a first rotational joint and configured to rotate about a yaw axis;
(c) a pitch mechanism operably coupled to the positioning body at a second rotational joint and configured to rotate about a pitch axis that intersects the yaw axis at an incision; and
(d) a plunge mechanism slidably coupled to the pitch mechanism such that the plunge mechanism can move along a plunge axis.
(a) a positioning body;
(b) a yaw mechanism operably coupled to the positioning body at a first rotational joint and configured to rotate about a yaw axis;
(c) a pitch mechanism operably coupled to the positioning body at a second rotational joint and configured to rotate about a pitch axis that intersects the yaw axis at an incision; and
(d) a plunge mechanism slidably coupled to the pitch mechanism such that the plunge mechanism can move along a plunge axis.

2. The gross positioning system of claim 1, wherein the plunge mechanism is configured to be coupleable to a robotic surgical device.

3. The gross positioning system of claim 1, wherein the yaw mechanism is configured to rotate the robotic surgical device about an incision without changing a pitch angle or plunge distance of the robotic surgical device with respect to the incision.

4. The gross positioning system of claim 1, wherein the yaw mechanism further comprises a motor operably engaged with an output shaft configured to rotate the positioning body around the first rotational joint.

5. The gross positioning system of claim 4, wherein the yaw mechanism further comprises:
(a) a drive gear coupled to the motor;
(b) a driven gear operably engaged with the drive gear;
(c) a screw coupled to the driven gear; and
(d) a wheel is coupled to the output shaft, wherein the wheel is operably engaged with the screw.

6. The gross positioning system of claim 1, wherein the pitch mechanism further comprises a motor operably engaged with a curved output rail configured to rotate the plunge mechanism around the second rotational joint.

7. The gross positioning system of claim 6, wherein the pitch mechanism further comprises:
(a) a screw coupled to the motor;
(b) a wheel operably engaged with the screw; and
(c) a rotatable gear operably coupled to the wheel, wherein the rotatable gear is operably engaged with the curved output rail.

8. The gross positioning system of claim 1, wherein the plunge mechanism further comprises a motor operably engaged with an elongate output rail configured to translationally move the plunge mechanism along the plunge axis.

9. The gross positioning system of claim 1, wherein the plunge mechanism further comprises a clamp configured to be coupleable to the robotic surgical device.

10. The gross positioning system of claim 1, wherein a first axis of rotation of the first rotational joint, a second axis of rotation of the second rotational joint, and the plunge axis intersect at a single point of intersection.

11. The gross positioning system of claim 10, further comprising two or more lasers configured to emit light beams intersecting at the single point of intersection.

12. The gross positioning system of claim 1, further comprising a controller operably coupled to the gross positioning system and the robotic surgical device, wherein the gross positioning system and robotic surgical device are configured to operate together to position the robotic surgical device within a body cavity of a patient.

13. A gross positioning system for use with a robotic surgical device, the system comprising:
(a) a positioning body;
(b) a yaw mechanism operably coupled to the positioning body at a first rotational joint and configured to rotate about a yaw axis;
(c) a pitch mechanism operably coupled to the positioning body at a second rotational joint and configured to rotate about a pitch axis that intersects the yaw axis at an incision;
(d) a plunge mechanism slidably coupled to the pitch mechanism such that the plunge mechanism can move along a length of a plunge axis, wherein the plunge mechanism is configured to translationally move the robotic surgical device along the length of the plunge axis; and
(e) the robotic surgical device operably coupled to the plunge mechanism, the robotic surgical device comprising:
(i) a device body; and
(ii) an arm operably coupled to the device body, the arm comprising an end effector;
wherein the robotic surgical device is positionable through an insertion point in a patient such that the arm and at least a portion of the device body is positionable within a body cavity of the patient at a desired yaw angle, pitch angle, and plunge depth.

14. The gross positioning system of claim 13, wherein a first axis of rotation of the first rotational joint, a second axis of rotation of the second rotational joint, and the plunge axis intersect at a single point of intersection.

15. The gross positioning system of claim 14, wherein the single point of intersection is disposed at some point along a portion of the robotic surgical device.

16. The gross positioning system of claim 14, wherein the single point of intersection is disposed at an insertion point of a patient and the arm is partially disposed through the single point of intersection.

17. An external gross positioning system for use with an internal robotic surgical device, the system comprising:
(a) a support arm;
(b) a positioning body operably coupled to the support arm;
(c) a yaw mechanism operably coupled to the positioning body at a first rotational joint and configured to rotate about a yaw axis;
(d) a pitch mechanism operably coupled to the positioning body at a second rotational joint and configured to rotate about a pitch axis that intersects the yaw axis at an insertion point; and
(e) a plunge mechanism slidably coupled to the pitch mechanism such that the plunge mechanism can move along a length of a plunge axis, wherein the plunge mechanism is configured to be coupleable to the internal robotic surgical device.

18. The external gross positioning system of claim 17, wherein the support arm further comprises:
(a) a clamp configured to couple with a bed rail;
(b) a rod coupled to the clamp;
(c) a first elongate arm operably coupled to the rod at a third rotational joint; and
(d) a second elongate arm operably coupled to the first elongate arm at a fourth rotational joint and operably coupled to the positioning body at a fifth rotational joint.

19. The external gross positioning system of claim 18, wherein the third rotational joint, the fourth rotational joint, and the fifth rotational joint are each configured to rotate around parallel axes.

20. The external gross positioning system of claim 17, wherein the insertion point is a single point of intersection of the yaw axis, the pitch axis, and the plunge axis.
